(19) United States
(12) Patent Application Publication
(10) Pub. No.: US 2026/0151063 A1
(43) Pub. Date: Jun. 4, 2026

(54) METHODS AND SYSTEMS FOR MONITORING BIO-MAGNETIC SIGNALS

(71) Applicant: Ohio State Innovation Foundation, Columbus, OH (US)

(72) Inventors: Asimina KIOURTI, Columbus, OH (US); Keren ZHU, Columbus, OH (US)

(21) Appl. No.: 19/125,106

(22) PCT Filed: Oct. 27, 2023
(86) PCT No.: PCT/US2023/078051

§ 371 Date: Apr. 28, 2025

Related U.S. Application Data
(60) Provisional application No. 63/420,285, filed on Oct. 28, 2022.

(51) Int. Cl. A61B 5/16; A61B 5/00; A61B 5/024; A61B 5/243; A61B 5/352

(52) U.S. Cl. CPC A61B 5/165; A61B 5/02405; A61B 5/243; A61B 5/352; A61B 5/6802

(57) ABSTRACT

An example method for determining cognitive load is described herein. The method includes receiving a cardiac signal measured by a non-contact sensor; determining a heart rate variability (HRV) metric based on the cardiac signal; and determining a cognitive load of a subject based on the HRV metric. An example system for determining cognitive load is also described herein. The system includes a non-contact sensor; a computing device operably coupled to non-contact sensor, the computing device including a processor and a memory, the memory having instructions thereon, that, when executed, cause the processor to: receive a cardiac signal measured by a non-contact sensor; determine a heart rate variability (HRV) metric based on the cardiac signal; and determine a cognitive load of a subject based on the HRV metric.

### CROSS-REFERENCE TO RELATED APPLICATIONS

This application claims the benefit of U.S. provisional patent application No. 63/420,285, filed on Oct. 28, 2022, and titled “METHODS AND SYSTEMS FOR MONITORING BIO-MAGNETIC SIGNALS”, the disclosure of which is expressly incorporated herein by reference in its entirety.

### BACKGROUND

Heart rate variability is the variation in time intervals between heartbeats. Heartbeats can be measured using ECG sensors to detect the heart's electrical activity. ECG sensors may require a clean contact with the skin to be accurate, and therefore can be ineffective when used on surfaces of the body that include hair, sweat, dirt, clothing, and other obstructions. For example, some ECG can require 12-leads to be applied to the chest of a subject. Systems and methods for sensing heart rate variability using non-contact sensors can improve heart measurements.

### SUMMARY

Methods and systems for determining the cognitive load of a subject based on heart rate variability are disclosed herein.

A method for determining the cognitive load of a subject is described herein. The method includes receiving a cardiac signal measured by a non-contact sensor; determining a heart rate variability (HRV) metric based on the cardiac signal; and determining a cognitive load of a subject based on the HRV metric.

In some implementations, the non-contact sensor is a magnetocardiography (MCG) sensor. Alternatively or additionally, the non-contact sensor is a wearable sensor.

In some implementations determining the HRV metric includes evaluating the cardiac signal in a time domain. Optionally, the HRV metric is a standard deviation of RR intervals (SDRR) in the cardiac signal, a root mean square of successive differences between heartbeats (RMSSD) in the cardiac signal, or a mean value of adjacent R-peaks (MeanRR) in the cardiac signal. Alternatively or additionally, determining the HRV metric includes evaluating the cardiac signal in a frequency domain. Optionally, determining the cognitive load of the subject includes evaluating the HRV metric using a Poincaré plot.

In some implementations, the non-contact sensor includes a belt and a plurality of coils, wherein the coils are embedded in the belt.

In some implementations, determining the cognitive load of the subject includes distinguishing between high and low cognitive load based on the HRV metric. Alternatively or additionally, determining the cognitive load of the subject includes classifying the subject into one of a plurality of cognitive load categories based on the HRV metric. Alternatively or additionally, determining the cognitive load of the subject includes quantifying the cognitive load of the subject based on the HRV metric.

In some implementations, determining the HRV metric includes identifying R-peaks, identifying a plurality of MCG cycles using the R-peaks, and averaging the plurality of MCG cycles to obtain an MCG waveform.

A system for determining the cognitive load of a subject is described herein. The system includes a non-contact sensor; a computing device operably coupled to non-contact sensor, the computing device comprising a processor and a memory, the memory having instructions thereon, that, when executed, cause the processor to: receive a cardiac signal measured by a non-contact sensor; determine a heart rate variability (HRV) metric based on the cardiac signal; and determine a cognitive load of a subject based on the HRV metric.

In some implementations, the non-contact sensor is a magnetocardiography (MCG) sensor. Alternatively or additionally, the non-contact sensor is a wearable sensor.

In some implementations, determining the HRV metric includes evaluating the cardiac signal in a time domain. Optionally, the HRV metric is a standard deviation of RR intervals (SDRR) in the cardiac signal, a root mean square of successive differences between heartbeats (RMSSD) in the cardiac signal, or a mean value of adjacent R-peaks (MeanRR) in the cardiac signal. Alternatively or additionally, determining the HRV metric includes evaluating the cardiac signal in a frequency domain.

In some implementations, determining the cognitive load of the subject includes evaluating the HRV metric using a Poincaré plot.

In some implementations, the non-contact sensor includes a belt and a plurality of coils, wherein the coils are embedded in the belt.

In some implementations, determining the cognitive load of the subject includes distinguishing between high and low cognitive load based on the HRV metric. Alternatively, or additionally, determining the cognitive load of the subject includes classifying the subject into one of a plurality of cognitive load categories based on the HRV metric. Optionally, determining the cognitive load of the subject includes quantifying the cognitive load of the subject based on the HRV metric.

In some implementations, determining the HRV metric includes identifying R-peaks, identifying a plurality of MCG cycles using the R-peaks, and averaging the plurality of MCG cycles to obtain an MCG waveform.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates an example method for determining cognitive load of a subject based on a heart rate variability metric.

FIG. 2 illustrates an example system block diagram of a system for determining cognitive load of a subject based on a heart rate variability metric.

FIG. 3 illustrates an example system and example experimental results.

FIG. 4 is an example computing device.

FIG. 5 illustrates a study of an experimental implementation of the present disclosure.

FIG. 6 illustrates the participants used in a study of the example implementation of the present disclosure.

FIG. 7 illustrates a comparison of MCG and ECG signals recorded on a human participant for the same time duration, showing that two R-peaks were detected in each case, validating the ability of the example implementation of an MCG sensor to monitor HRV parameters.

FIG. 8 illustrates a table including MCG and ECG derived HRV parameters for a human participant across 7 trials, according to an example implementation of the present disclosure.

FIG. 9 illustrates a summary of study participants' self-reported level of difficulty and math performance.

FIG. 10A illustrates the SDRR metric for study subjects using the MCG sensors.

FIG. 10B illustrates the SDRR metric for study subjects using the ECG sensors.

FIG. 10C illustrates the RMSSD metric for subjects using the MCG sensors.

FIG. 10D illustrates the RMSSD metric for study subjects using the ECG sensors.

FIG. 10E illustrates the MeanRR metric for study subjects using the MCG sensors.

FIG. 10F illustrates the MeanRR metric for study subjects using the ECG sensors.

FIG. 11A illustrates SDRR of a subject using MCG.

FIG. 11B illustrates SDRR of a subject using ECG.

FIG. 11C illustrates RMSSD of a subject using MCG.

FIG. 11D illustrates RMSSD of a subject using ECG.

FIG. 11E illustrates MeanRR of a subject using MCG.

FIG. 11F illustrates of a subject MeanRR using ECG.

FIG. 12A illustrates an example recording system, according to implementations of the present disclosure.

FIG. 12B illustrates a block diagram of sensor and electronics for the example recording system illustrated in FIG. 12A.

FIG. 13A illustrates an in-vivo experimental setup including an example implementation of the present disclosure.

FIG. 13B illustrates an MCG confirmation test setup including an example implementation of the present disclosure.

FIG. 14A illustrates MCG confirmation data including cycle-averaged MCG over time recorded at away from chest, according to an example implementation of the present disclosure.

FIG. 14B illustrates validation test data in earth ambient noise according to an example implementation of the present disclosure.

FIG. 14C illustrates an example validation test including cycle-averaged MCG over ˜4.5 minutes.

FIG. 15 illustrates a table including intra-subject QRS detection accuracy, according to an example implementation of the present disclosure.

FIG. 16 illustrates a table including inter-subject QRS detection accuracy, according to an example implementation of the present disclosure.

FIG. 17 illustrates intra-subject averaged R-R interval and R-R accuracy, according to an example implementation of the present disclosure.

FIG. 18 illustrates inter-subject averaged R-R interval and R-R accuracy, according to an example implementation of the present disclosure.

FIG. 19 illustrates RMS of isoelectric regions comparing no movement vs. minimal movement, according to an example implementation of the present disclosure.

FIG. 20 illustrates minimal averaging time comparing no movement vs. minimal movement, according to an example implementation of the present disclosure.

FIG. 21A illustrates an example of a processed averaged MCG using the minimal average time from a subject in a study of an example implementation of the present disclosure with no movement.

FIG. 21B illustrates an example of a processed averaged MCG using the minimal average time from a subject in a study of an example implementation of the present disclosure with minimal movement.

FIG. 21C illustrates an example of a processed averaged MCG using the minimal average time from a subject in a study of an example implementation of the present disclosure with averaging over 13 cardiac cycles in a minimal movement study, where the same minimal cycles were used in a no-movement study.

FIG. 22A illustrates cycle-averaged MCG vs. ECG over 4.5 minutes, according to an example implementation of the present disclosure.

FIG. 22B illustrates cycle-averaged MCG when converted to magnetic field units (Tesla) according to an example implementation of the present disclosure.

FIG. 23A illustrates a converted magnetic field plot showing MCG confirmation of cycle-averaged MCG over ˜1 min of time recorded at 13 cm away from the chest, according to an example implementation of the present disclosure.

FIG. 23B illustrates a validation test plot showing real-time MCG vs. ECG in earth ambient noise, according to an example implementation of the present disclosure.

FIG. 24 illustrates time widths of QRS, PQ, and QT in ˜5 min averaged MCG for a no-movement study.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for tracking, monitoring, or determining cognitive load using non-contact sensors, it will become evident to those skilled in the art that the implementations are not limited thereto, but are applicable for measuring heart rate variability using non-contact sensors, or measuring any other magnetic or electric signal using non-contact sensors. This may include, but is not limited to, a consumer device such as a health accessory used to track the cognitive load or health status of a subject.

Heart rate variability (HRV) is a useful measurement for characterizing the performance of the heart of a subject. HRV refers to the variation in the time interval between heartbeats and correlates to activities of both the autonomic nervous system (ANS) and the cardiovascular system. For example, HRV can be correlated to cognitive performance, heart health, sleep, stress, and athletic performance. In particular, there are benefits to wearable devices that can measure HRV, including wearable devices that can measure HRV in real time.

Conventional HRV measurement devices include electrocardiogram (ECG) sensors. ECG sensors commonly use electrodes to measure electrical signals on the skin of an individual. Many ECG sensors require direct electrode contact with the skin which makes them cumbersome for daily wear, have low signal/contact quality when employed outside the clinical environment, prone to errors with underlying sweat and hair, and/or may cause skin irritation and allergies (which, in turn, degrade signal quality). Thus, conventional HRV measurement devices using ECG sensors are not suitable for use as wearable devices, and are limited to clinical contexts.

Implementations of the present disclosure include improved magnetocardiography (MCG) sensors configured to accurately quantify heart rate variability. Systems, methods, and devices using MCG sensors can measure HRV without requiring direct skin contact to the sensors. Thus, MCG sensors can be used in wearable devices where ECG sensors would be ineffective. Implementations of the present disclosure can therefore be used to measure HRV outside of clinically controlled contexts.

For example, implementations of the present disclosure include systems, devices, and methods for wearable devices that determine the cognitive load of an individual based on the HRV of that individual. Implementations of the present disclosure include MCG sensors that can include an array of miniaturized coils that couple to the magnetic field of the heart when placed in proximity (e.g., upon the chest). The resulting signal can be post-processed to denoise the collected signals (e.g., averaging across the coils, filtering, etc.), the R-peaks can be retrieved. The example implementation of an MCG sensor does not suffer from the skin-contact-related issues for ECG and can be comfortably worn as part of a garment, making it a solution for real-world monitoring of HRV, and therefore for other parameters that correlate with HRV like cognitive workload. MCG signals are transparent to underlying tissues (tissues are non-magnetic), providing promise for even better accuracy as compared to ECG-based metrics (tissues are dielectric materials impacting the electric field).

It should be understood that determining cognitive load is only an example use case of the systems and methods described herein using non-contact sensors to receive cardiac signals. Implementations of the present disclosure can be used to perform real-time MCG on subjects using non-contact sensors (e.g., coils) for any purpose. Example implementations of performing real-time MCG with non-contact sensors are described with reference to Example 2.

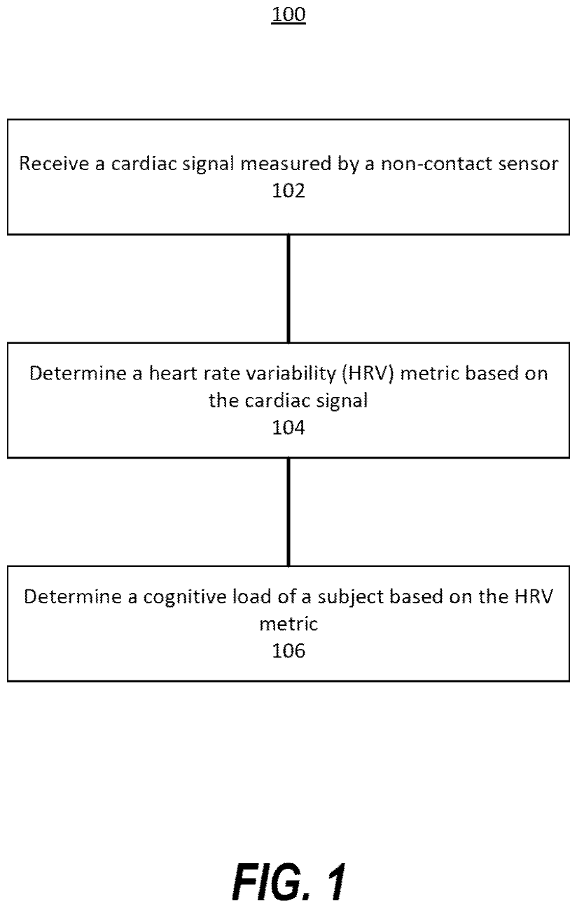

With reference to FIG. 1, the present disclosure includes systems and methods for measuring heart rate variability and/or determining cognitive load using non-contact sensors. As used herein, “non-contact” means that the sensor can operate without sensor contact with the skin. For example, a sensor that is embedded in a wearable device is a non-contact sensor if the wearable device touches the skin of a user, but the sensor embedded in the wearable device does not touch the skin, or is not required to touch the skin to sense a signal from the wearer of the wearable device.

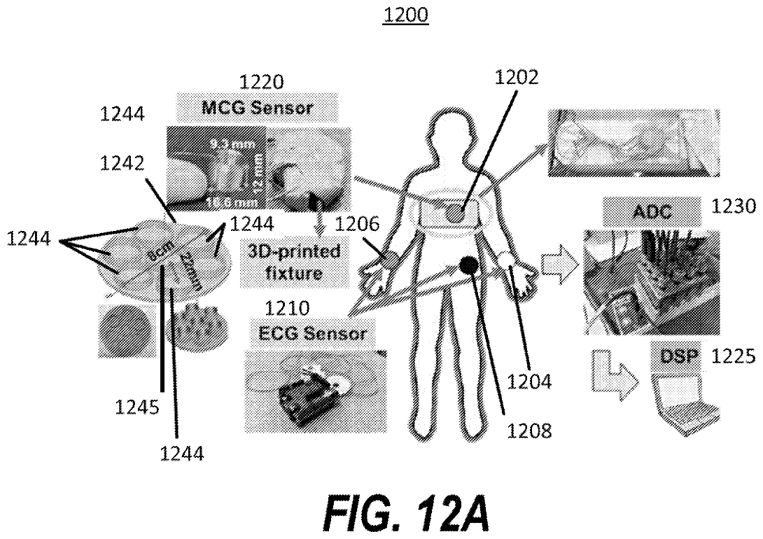

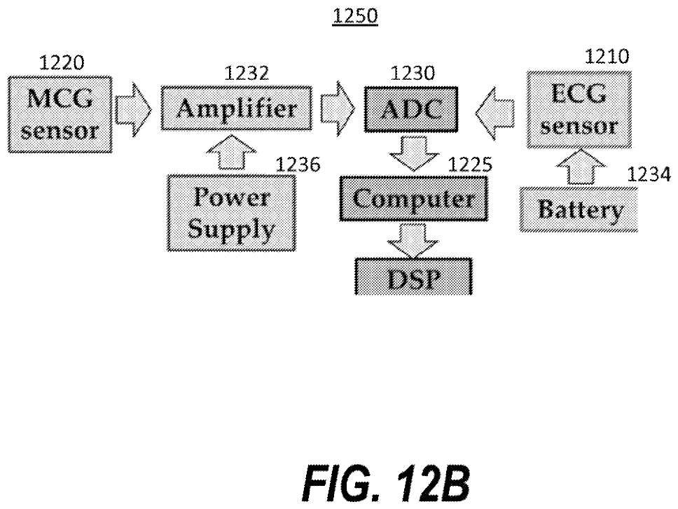

Still with reference to FIG. 1, an example method 100 for determining the cognitive load of a subject is shown. At step 102, a cardiac signal is received, where the cardiac signal is a cardiac signal measured by a non-contact sensor. The non-contact sensor can be a non-contact sensor that includes any number of coils. Coils 1246 are shown and described more detail with reference to FIG. 12A and Examples 1 and 2.

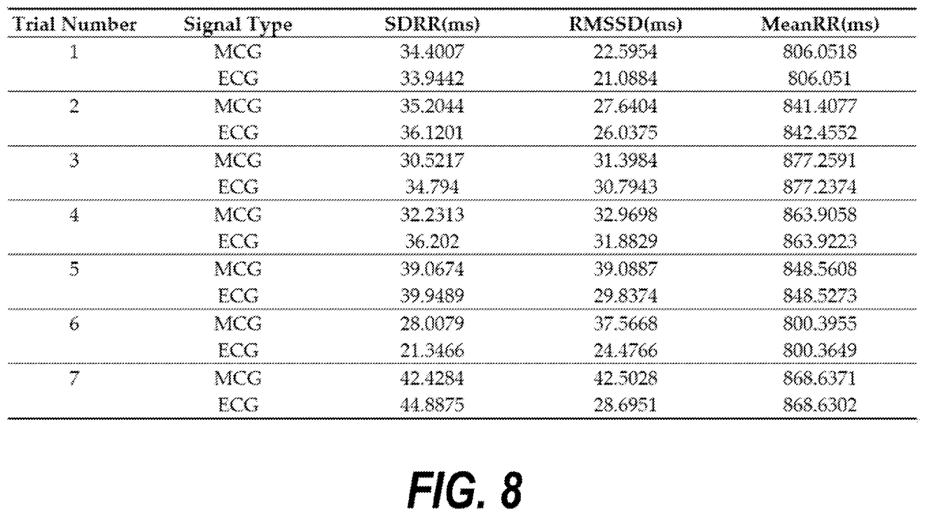

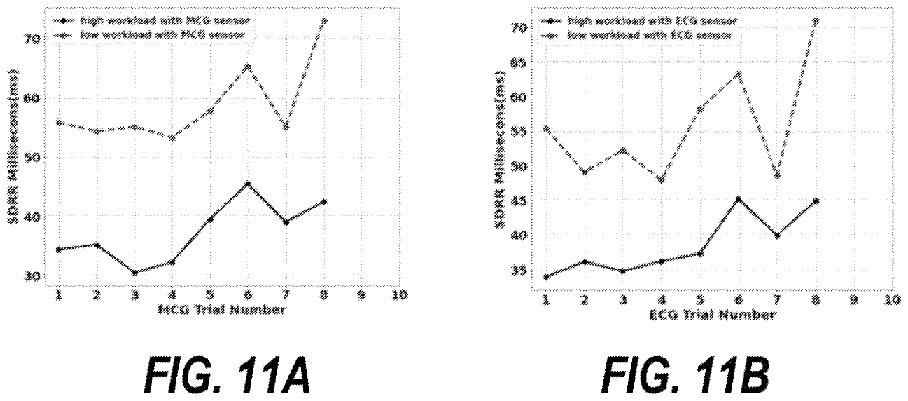

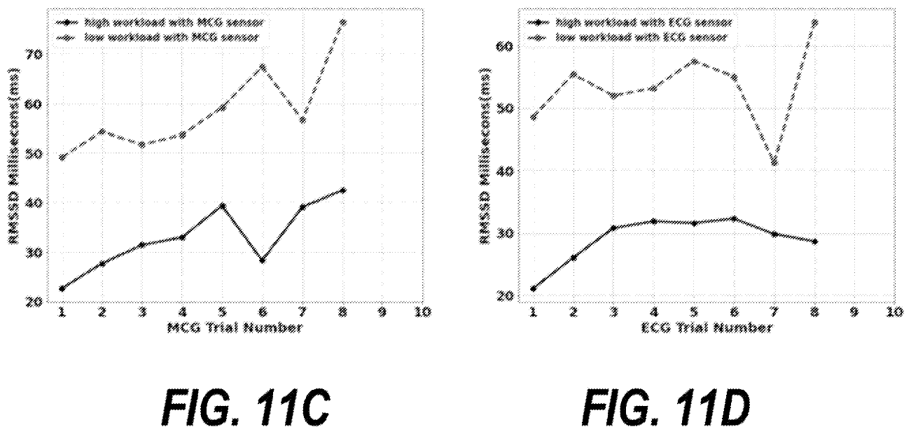

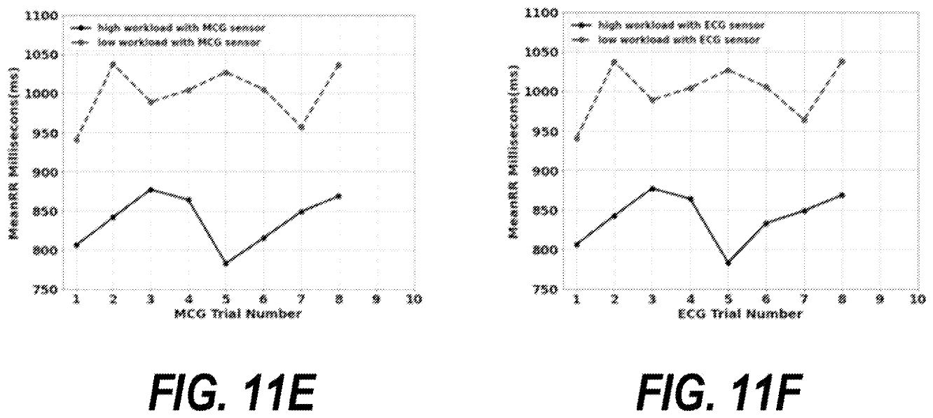

At step 104, a heart rate variability metric is determined based on the cardiac signal received at step 102. In some implementations, the heart rate variability metric is determined by evaluating the cardiac signal in a time domain and/or evaluating the cardiac signal in the frequency domain. Heart rate variability metrics are also referred to herein as “HRV parameters.” Alternatively or additionally, in some implementations, the heart rate variability metric is determined using a standard deviation of RR intervals (SDRR) in the cardiac signal, a root mean square of successive differences between heartbeats (RMSSD) in the cardiac signal, or a mean value of adjacent R-peaks (MeanRR) in the cardiac signal. Additional examples of determining heart rate variability metrics and HRV parameters are described herein, for example in Example 1 (e.g., FIG. 8), and Example 2 (e.g., FIGS. 11A-11F).

In some implementations, the HRV metric can be evaluated using a Poincaré plot.

At step 106, a cognitive load of a subject is determined based on the heart rate variability metric determined at step 104. In some implementations, the cognitive load of the subject can be determined by distinguishing between high and low cognitive load based on the HRV metric determined at step 104. Cardiovascular activity relates to human cognitive functioning. The amount of cognitive effort exerted by an individual is referred to herein is referred to as “cognitive load.” HRV indexes (e.g., indexes including the HRV metrics determined at step 104) can be used to quantify the cognitive workload of subjects. For example, mathematical matrices can be used to evaluate HRV including using time domain analysis, frequency domain analysis, and Poincaré plots. Implementations of the present disclosure can estimate cognitive load of a subject based on the relationships between HRV and cognitive load described herein (e.g., the study of an example implementation of the present disclosure described in Example 1).

Alternatively or additionally, in some implementations, the cognitive load can be determined by classifying the subject into one of a plurality of cognitive load categories based on the HRV metric. As described in greater detail in example 1, herein, classifying the subject into one of the cognitive load categories based on the HRV metric can include determining whether the subject is in a high cognitive load state or a low cognitive load state. A non-limiting example of a high cognitive load state is the cognitive load of performing arithmetic, and a low cognitive load state is the cognitive load of watching a relaxing video. And, alternatively or additionally, determining the cognitive load of the subject can include quantifying the cognitive load of the subject based on the HRV metric.

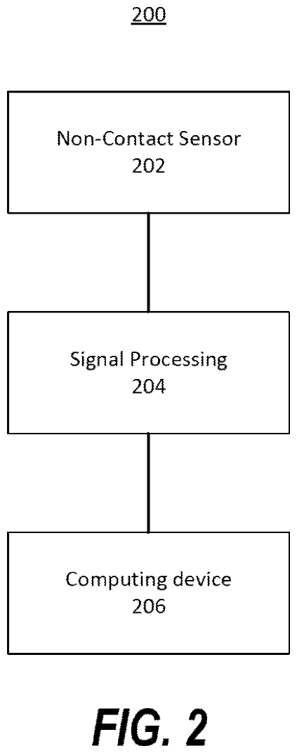

With reference to FIG. 2, implementations of the present disclosure include systems for determining cognitive load based on heart rate variability. The system 200 can include a non-contact sensor 202, a signal processing module 204, and a computing device 206.

The non-contact sensor 202 be a magnetocardiography sensor, as described with respect including one or more coils. Examples of coils that can be used in an example magnetocardiography sensor are described with reference to the coils 1246 shown in FIG. 12A and described in examples 1 and 2.

In some implementations, the non-contact sensor 202 is a wearable sensor that can be configured to be integrated into a wearable device. Non-limiting examples of wearable devices include watches, chest straps, and any type of clothing. As a further non-limiting example, the non-contact sensor 202 can include a belt and one or more of coils, where the coils are embedded in the belt so that the coils do not touch the skin of the user when the user wears the belt.

In some implementations, the non-contact sensor 202 is a magnetocardiography sensor configured to output a cardiac signal. A magnetocardiography sensor can include one or more coils, where the coils are configured to detect magnetocardiographic signals from a subject's heart without touching the skin of the subject. The magnetocardiography sensor(s) can be integrated into one or more wearable devices.

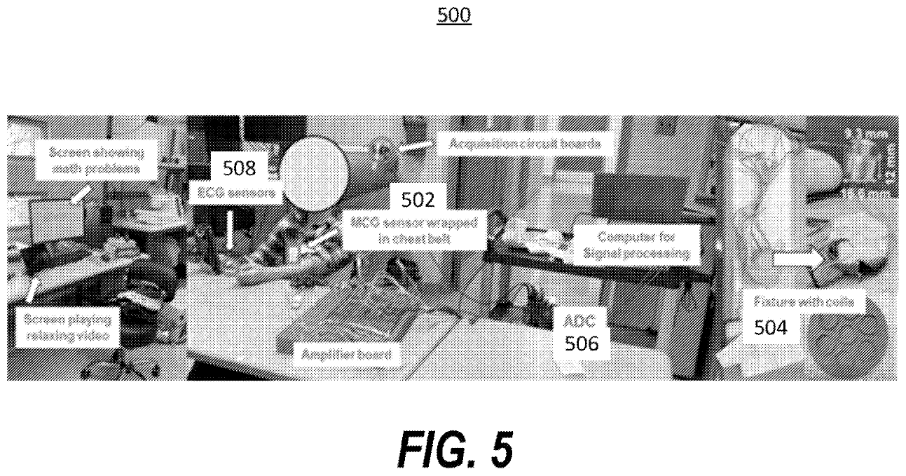

The non-contact sensor 202 can be operatively connected to a signal processing module 204. The signals from the non-contact sensor can be processed using a signal processing module 204. The signal processing module can include analog to digital converters, amplifiers, filters (band pass, low pass, high pass, etc.). The operation of the analog to digital converters and other example signal processing techniques are described in greater detail in examples 1 and 2, for example with reference to the ADC 506 shown in FIG. 5, and the ADC 1230 shown in FIG. 12A In implementations where the non-contact sensor outputs a cardiac signal, the signal processing module can be configured to amplify the signal, filter noise from the signal, and convert the signal into an analog or digital output.

The output of the signal processing module 204 can be operatively connected to a computing device 206. In some implementations, the computing device 206 can be configured to determine the cognitive load of the subject based on a heart rate variability metric, for example by performing the method described with respect to steps 104 and 106 of FIG. 1.

Additional example systems and methods for determining cognitive load are described herein with reference to Example 1, including FIG. 5, for example. Additional example systems and methods for magnetocardiography are described herein with reference to Example 2. Additional example systems and methods for determining heart rate variability metrics are described herein with reference to the “experimental implementation.”

### Experimental Implementation

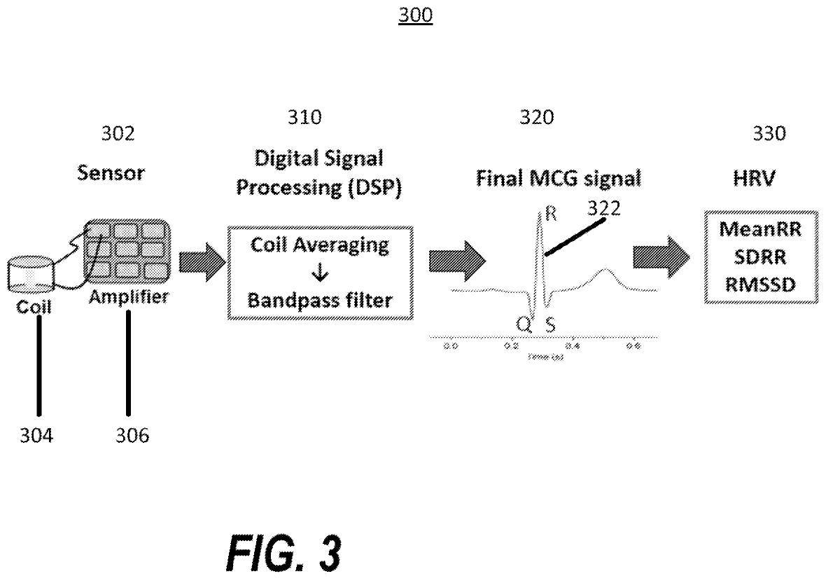

FIG. 3 illustrates an experimental implementation 300 of the present disclosure used to generate heart rate variability metrics. An example sensor 302 is shown including a coil 304 and an amplifier 306 configured to amplify a signal output by the coil 304. It should be understood that the experimental implementation can include any number of coils 304 and amplifiers 306.

The output of the amplifiers 306 can be connected to a digital signal processing stage 310. The digital signal processing stage 310 can average the outputs of the amplifier(s) 306 and filter the outputs of the amplifier(s) 306. The output of the digital signal processing stage 310 can include a magnetocardiography signal. An example waveform 322 of a magnetocardiography signal is shown in the plot 320.

The magnetocardiography signal can be processed to determine heart rate variability data 330. In the experimental implementation, the heart rate variability data 330 includes mean RR (mean time between heart beats); SDRR (standard deviation of time between heart beats); and RMSSD (root mean square successive RR interval differences).

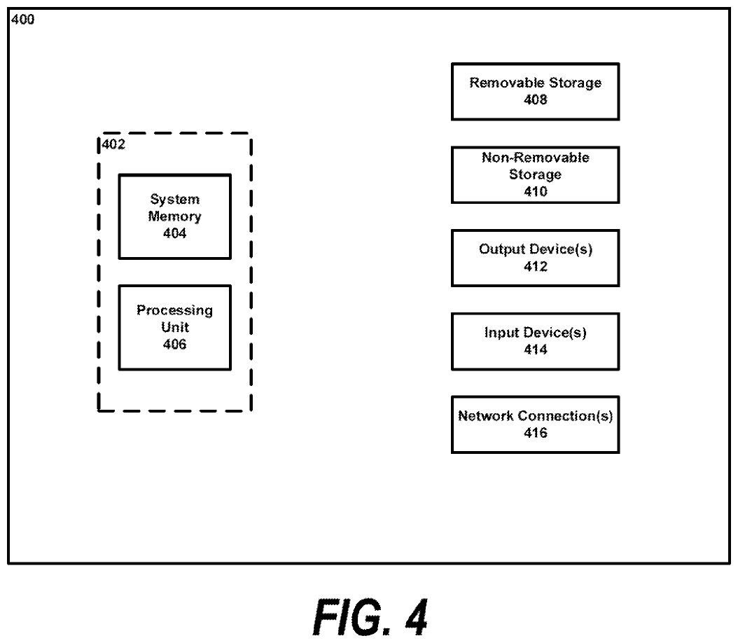

It should be appreciated that the logical operations described herein with respect to the various figures may be implemented (1) as a sequence of computer implemented acts or program modules (i.e., software) running on a computing device (e.g., the computing device described in FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by dashed line 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example 1: Quantifying Cognitive Workload Using a Non-Contact Magnetocardiography (MCG) Wearable Sensor

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

A study was performed using an example implementation of the present disclosure. The example implementation of the present disclosure was configured to quantify cognitive workload. As used herein, “cognitive workload” can refer to the level of mental effort put forth by an individual in response to a cognitive task. Cognitive workload can be relevant for healthcare, training, and gaming applications. Implementations of the present disclosure can be used to readily and reliably quantify cognitive workload of an individual in a real-world environment at a seamless form factor and affordable price.

The study described herein demonstrates the feasibility of a magnetocardiography (MCG) sensor and system according to the present disclosure, used to reliably classify high vs. low cognitive workload while being non-contact, fully passive, low-cost, and with the potential to have a wearable form factor. The operating principle measures the emanated magnetic fields from the heart and analyzes the heart rate variability (HRV) matrix in three time-domain parameters: standard deviation of RR intervals (SDRR), root mean square of successive differences between heartbeats (RMSSD), and mean values of adjacent R-peaks in the cardiac signals (MeanRR). In vivo experimental results show that SDRR and RMSSD achieve 100% accuracy in classifying high vs. low cognitive workload in 11 participants, while MeanRR achieves 91% accuracy. Tests for the same individual yield an intra-subject classification accuracy of 100% for all three HRV parameters.

Cognitive workload is defined as the level of mental effort put forth by an individual in response to a cognitive task [1]. For example, if considerable mental effort is exerted, the cognitive workload is high and the individual's information-processing abilities may slow down [2]. Quantifying the level of cognitive workload in real-time can be relevant to several applications. Non-limiting examples include preventing distracted driving [3], rating pilots' performance [4], and providing individualized return-to-learn guidelines following mild traumatic brain injury [5]. Though questionnaire surveys and observation of human behavior can be used to estimate cognitive workload, such estimates are subjective (hence, inaccurate) and not available on a continuous, real-time basis [6, 7, 8].

Measurements of various physiological signals from the human body can be used to objectively reflect cognitive workload changes. For example, electroencephalography (EEG) has emerged as a promising technology in this regard, where brain oscillations in the alpha and theta bands are sensitive to the mental task difficulty level [9]. Specifically, as the task difficulty increases, the cognitive workload increases, so that alpha waves become weaker whereas theta waves become stronger [10]. However, EEG devices are obtrusive, consisting of tens of electrodes placed around the scalp as well as heavy amplifiers and cable connections to recording devices [11]. As such, they can be undesirable for monitoring an individual's cognitive workload in a natural environment. Portable EEG headsets with a small number of channels can output signals with a noise level that is high enough to prohibit highly accurate monitoring of cognitive workload in real-world scenarios. Furthermore, placing electrodes on the scalp takes time, causes discomfort, and provides no guarantee for acceptable signal quality over long periods of time. Pupillometry can be another means of quantifying cognitive workload via ‘smart’ glasses that monitor pupil dilation [13]. However, ambient light can act as a contaminating component that influences pupil dilation and there are very few approaches available to reduce that noise [14]. Additionally, the expense of ‘smart’ glasses can make them unsuitable for day-to-day use.

Electrocardiograman accurately classify workload based on cardiac measures and, specifically, heart rate variability (HRV) [15]. HRV quantifies the variation in the time interval between consecutive heartbeats and correlates to activities of both the autonomic nervous system (ANS) and the cardiovascular system [16, 17]. HRV can exhibit sensitivity to task load, conditions of event rate, and task duration. For example, HRV extracted from ECG can successfully differentiate between different flight and driving phases. [18, 19] In [20], HRV achieved a classification accuracy of 93.4% in detecting high cognitive load and >90% in detecting real-life stress. However, ECG sensors require direct electrode contact with the skin which makes them cumbersome for daily wear, have low signal/contact quality when employed outside the clinical environment, are prone to errors with underlying sweat and hair, and may cause skin irritation and allergies (which, in turn, degrade signal quality). [21, 22].

The study described herein demonstrates the feasibility of a magnetocardiography (MCG) sensor to accurately quantify cognitive workload while overcoming limitations in the state-of-the-art. MCG is the magnetic field equivalent of ECG: it measures the magnetic flux induced by the current flowing through the heart. An MCG sensor can capture the R-peaks of cardiac activity in real-time, in a non-contact manner, i.e., without any skin contact [23]. The example sensor can include an array of miniaturized coils that couple to the magnetic field of the heart when placed in proximity (e.g., upon the chest). Following extensive post-processing to denoise the collected signals (including averaging across the coils, filtering, etc.), the R-peaks can be retrieved. The example implementation of an MCG sensor does not suffer from the skin-contact-related issues described earlier for ECG and can be comfortably worn as part of a garment, making it a highly promising solution for real-world monitoring of cognitive workload [24]. An added advantage is that MCG signals are transparent to underlying tissues (tissues are non-magnetic), providing promise for even better accuracy as compared to ECG-based metrics (tissues are dielectric materials impacting the electric field). The study of the example implementation reports a proof-of-concept study to confirm the MCG sensor's feasibility in this regard. The study recruited 11 healthy adults of ages 20 to 35, monitored the MCG-derived HRV indexes as they performed low and high cognitive workload activities, and demonstrated successful classification of the cognitive workload level at 91% to 100% accuracy for different HRV metrics and 100% accuracy for multiple repetitions on the same subject. The example implementation shows that an MCG sensor can be effective for cognitive workload classification.

FIG. 5 illustrates an example experimental setup 500 for the study of the present example. Each participant was asked to sit on a zero-gravity chair with an MCG sensor 502 wrapped around his/her chest. The chair was selected to provide comfort across the duration of the experiment and reduce motion artifacts for the study. The MCG sensor 502 was designed to include an array of seven coils (each 11 mm in height and 15 mm in diameter), embedded within a circular 3D-printed fixture of 60 mm in diameter. [23, 24]. The fixture 504 with the embedded coils is shown in FIG. 5, and was embedded in an elastic chest belt that was wrapped around the participant's torso. The study counted from the clavicle and down to the space between the third and fourth ribs to identify the location of the heart, and aligned the MCG sensor with this location [25]. Raw MCG signals were captured by the human heart, bandpass filtered, amplified, digitized via an Analog to Digital Converter (ADC) 506, and sent to a laptop computer for post-processing. To collect “gold-standard” cardiac measures for comparison, a 3-lead ECG sensor 508 was attached to the participant's skin. The ECG electrodes were placed on the abdominal area, left wrist, and right wrist, respectively. ECG data travelled from the leads to an acquisition circuit board and then eventually to the ADC and the laptop computer. The ADC sampling rate for both MCG and ECG was set to 5 kHz. To induce low and high cognitive workload, two screens were placed in front of the participant, as will be described in detail with reference to the present example.

The study included low and high cognitive workload test conditions. To induce low cognitive workload in the study, the participants were asked to watch a relaxing video on the screen placed in front of them. To induce high cognitive workload, the participants were asked to perform a dual-task which required the participants to watch the same relaxing video while simultaneously providing an answer to simple math problems of 2 digits±2 digits (i.e., addition or subtraction of 2-digit numbers). The math problems were shown on the screen of a tablet in front of the participants along with an answer. The participants were instructed to tap “1” on a tablet if they believed the answer was correct and “0” if it was incorrect. All math problems were machine-based and appeared automatically on the screen of a tablet one by one with an interval of 1.5 seconds. The participants' responses were recorded in parallel to their MCG and ECG signal activity to confirm their level of engagement. In summary, a mental arithmetic task was used to differentiate between low and high cognitive workload, as based on the sensory intake/rejection hypothesis previously reported in the literature [26]. Each scenario, i.e., low cognitive workload or high cognitive workload, lasted for 5 minutes according to previous studies for ECG-based workload classification [27]. At the end of each test session, the study asked each participant to report their perceived difficulty level of the math problems or their efforts to complete the math problem as difficult, not difficult nor easy, or easy to confirm that the high cognitive workload condition has been successfully induced.

The study further included Heart Rate Variability (HRV) Indexes. By monitoring the time intervals between consecutive R-peaks of the MCG and ECG signals, HRV parameters can be retrieved. As described herein, cardiovascular activity relates to human cognitive function and ECG-derived HRV indexes can be used to quantify the cognitive workload of drivers and pilots. Three example mathematical matrices can be used to evaluate HRV: time domain analysis, frequency domain analysis, and Poincaré plots [28, 29]. This study included pursued time domain analysis, and specifically evaluated the following three metrics: (a) standard deviation of RR intervals (SDRR), (b) root mean square of successive RR interval differences (RMSSD), and (c) mean of RR intervals (MeanRR). However, it should be understood that other evaluations of HRV (e.g., frequency domain analysis or Poincaré plots) can be used in other implementations of the present disclosure. The example implementation of the present disclosure used an R-peak algorithm to detect the maximum amplitude signal for each heartbeat of the ECG and MCG data [30] and then calculated the abovementioned metrics, ensuring that they lie within the anticipated ranges check for an example algorithm described herein (i.e., healthy SDRR should be 15.39 to 93 ms, mean RR should be 800 to 1300 ms, and RMSSD should be 15 to 75 ms [31, 32, 33, 34].

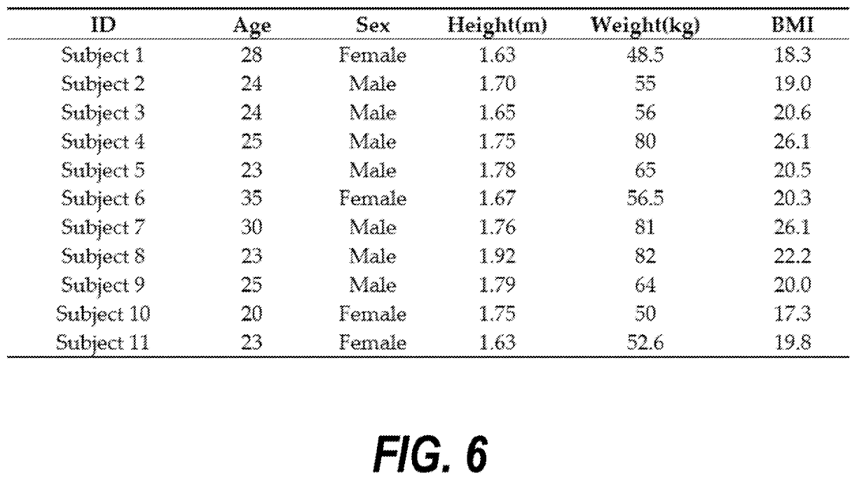

For the study, 11 healthy adults (4 females and 7 males) between the ages of 20 and 35 (M=25.45 years; SD=3.94 years) were recruited, as shown in FIG. 6. Data on sex were collected to confirm the sensor performance regardless of the presence of breast tissue. Age of participants was collected although it was not of relevance to this study. Height and weight data were collected and the Body Mass Index (BMI) was calculated to serve as an indicator of the distance between the MCG sensor and the heart. In this study, the study excluded participants with high BMI to reduce the risk of poor signal quality due to the weak strength of the MCG signal reaching the sensor.

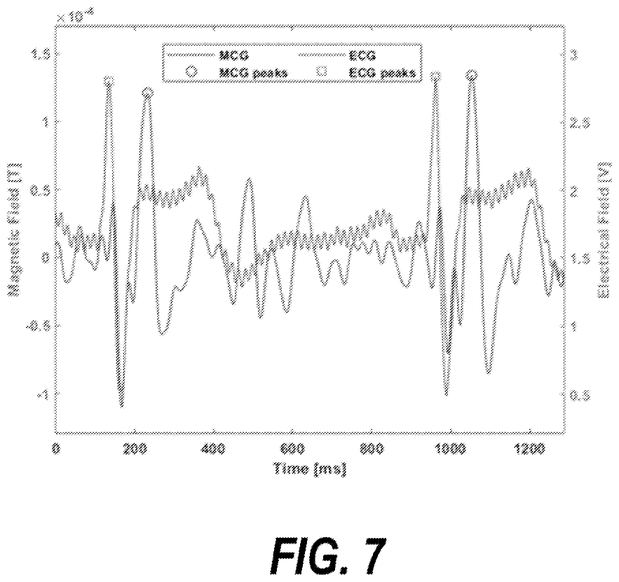

The study included validation of MCG sensor performance. The study validated the performance of the MCG sensor and its ability to accurately derive the target HRV indexes. The study recruited one participant and collected synchronized MCG and ECG data for a total of ˜3 minutes as described herein, without specifically inducing low or high cognitive workload conditions. To ensure repeatability, the study repeated this test seven (7) different times. An example plot of MCG and ECG data is shown in FIG. 7, confirming the intended correlation between the two plots. Specifically, the two signals were not expected to be in perfect synchronization given that MCG is the derivative of ECG, but the number of R-peaks and, hence, the number of QRS complexes can be the same [35]. Using the R-peak detection algorithm described herein, the study identified the R-peaks and calculated the SDRR, RMSSD, and MeanRR as summarized in FIG. 8. In the study all HRV values were within the anticipated ranges described herein, while the MCG-derived SDRR and MeanRR metrics were very close to those derived by “gold-standard” ECG. The MCG-derived RMSSD metric aligned well with the ECG-derived value for most trials but was different as encountered in the last three trials. This may be due to noise and/or RMSSD being known to be more sensitive to the parasympathetic nervous system (PNS) as compared to SDRR and MeanRR. [36].

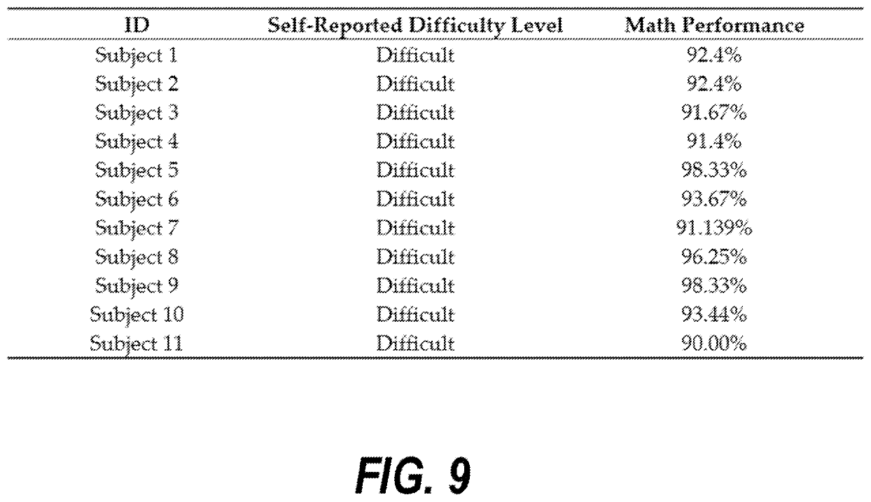

The study included confirmation of high cognitive workload testing conditions. The study recorded MCG and ECG data for the 11 participants of FIG. 9 during the low and high cognitive workload activities described in the present example. FIG. 9 summarizes: (a) the difficulty level of the math problems as self-reported by the participants, and (b) the participants' performance to the math problems (i.e., percentage of the answers they got right). As seen, all 11 subjects reported that the problems were “Difficult”, hence confirming that high cognitive workload was induced. Also, all subjects achieved ≥90% accuracy in answering the math problems, confirming that the participants engaged throughout the high cognitive workload task.

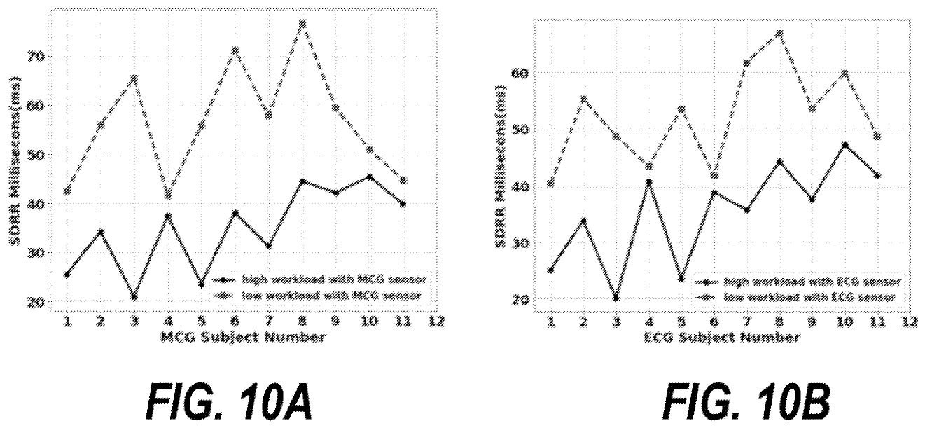

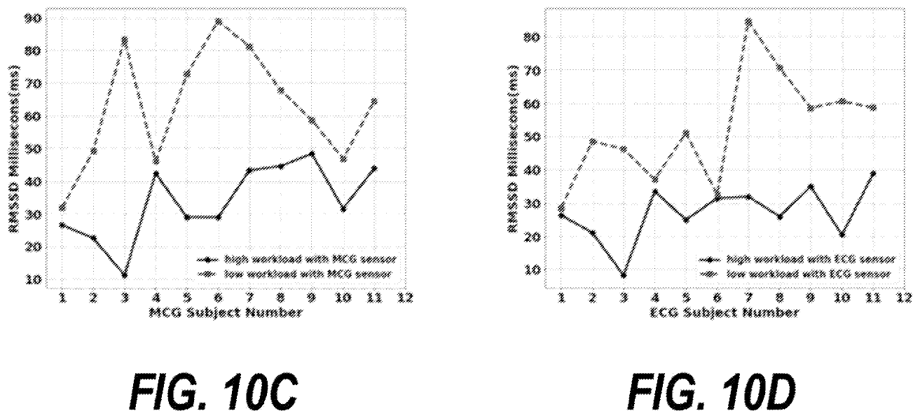

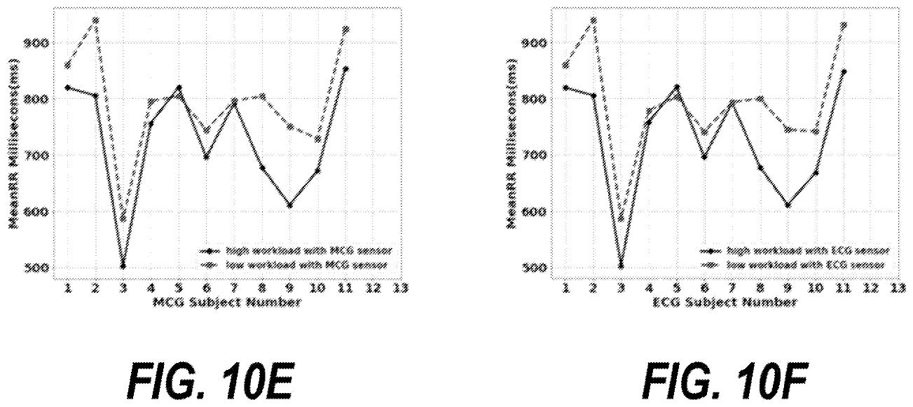

The study further included inter-subject classification performance. HRV parameters derived using the MCG and ECG sensors are shown in FIGS. 10A-10F. Specifically, FIG. 10A shows the SDRR metric for subjects using the MCG sensors. FIG. 10B shows the SDRR metric for subjects using the ECG sensors. FIG. 10C shows the RMSSD metric for subjects using the MCG sensors. FIG. 10D shows the RMSSD metric for subjects using the ECG sensors. FIG. 10E shows the MeanRR metric for subjects using the MCG sensors. FIG. 10F shows the MeanRR metric for subjects using the ECG sensors. The dashed line corresponds to the low cognitive workload condition, while the solid line corresponds to the high cognitive workload condition. FIGS. 10A-10F show that excellent performance was observed in distinguishing between high and low cognitive workload for the MCG and ECG sensors in the study of the example implementation. Notably, the classification accuracy of MCG was identical to “gold standard” ECG, confirming once again its reliability to monitor HRV. Specifically, SDRR achieved 100% accuracy in discerning low from high cognitive workload across the 11 subjects, RMSSD achieved 100% accuracy, and MeanRR achieved 91% accuracy. Specifically, the non-optimal accuracy for the MeanRR metric was due to Subject 5 who showed a higher instead of lower MeanRR value for the high cognitive workload case as compared to the low cognitive workload case. Environmental noise and respiratory frequency influences can impact the results of the example implementation.

Intra-Subject Classification Performance was also tested in the study. To confirm the intra-subject classification accuracy of the sensor, Subject 2 of FIG. 6 repeated the testing protocol eight (8) times on eight (8) different days. HRV results are summarized in FIGS. 11A-11F for the MCG- and ECG-derived metrics under low (dashed) and high (solid) workload conditions. FIGS. 11A-11F once again validates the performance of MCG as compared to “gold-standard” ECG and shows a 100% success rate of workload classification for this single participant. FIG. 11A illustrates SDRR using MCG. FIG. 11B illustrates SDRR using ECG. FIG. 11C illustrates RMSSD using MCG. FIG. 11D illustrates RMSSD using ECG. FIG. 11E illustrates MeanRR using MCG. FIG. 11F illustrates MeanRR using ECG.

The study described herein shows that an MCG sensor can be used to detect and classify cognitive workload in human participants. The results confirmed excellent agreement of HRV metrics (SDRR, RMSSD, MeanRR) derived using the MCG sensor as compared to “gold-standard” ECG. The results also confirmed the sensor's ability to distinguish between high and low cognitive workload using these HRV metrics.

The example implementation of an MCG sensor operates in non-shielded environments, does not require skin contact, and is low-cost hence overcoming limitations of state-of-the-art technologies used to classify cognitive workload. The electronics associated with the MCG sensor can be readily miniaturized for a wearable sensor form factor for operation in real-world environments.

The vivo studies demonstrated 100% success rate in classifying high vs. low cognitive workload for the SDRR and RMSSD metrics, and 91% success rate for the MeanRR metric across 11 participants. An additional two participants were recruited (both female) but demonstrated high noise in the collected MCG data, likely due to the presence of breast tissue that increased the distance between the heart and the sensor. They were hence excluded from the analysis. As is well known, the parasympathetic nervous system (PNS) is a division of the autonomic nervous system (ANS) that directly influences the increasing and decreasing HRV indexes. PNS will inhibit cardiac activities in response to increasing workload [37], hence it is expected that SDRR, RMSSD, and MeanRR will drop when people have higher attentional workload demands. This was confirmed by the results illustrated in FIGS. 10A-11F.

Repeatability results for a single participant showed 100% classification accuracy of high vs. low cognitive workload for all three HRV metrics under consideration. Though not generalizable, these results also showed a clear threshold level for each of the HRV metrics for the participant. This suggests that a personalized threshold likely exists for each individual, though subject to change over the course of time.

The study of the example implementation of an MCG sensor can be used for cognitive workload classification in numerous applications, both inside and outside laboratory settings. The present disclosure contemplates the use of additional HRV metrics, improving the sensor performance regardless of the presence of breast tissue, and automating cognitive workload classification using artificial intelligence, according to additional implementations of the present disclosure, and it should be understood that the study described herein is intended only as a non-limiting example.

### Example 2: Real-Time Magnetocardiography with Passive Miniaturized Coil Array in Earth Ambient Field

Implementations of the present disclosure include passive miniaturized coil arrays that can be used for monitoring real-time human MCG in a non-shielded environment. A second study was performed of an example implementation for real-time human MCG. The example implementation can include 7 individual coils (described with reference to example 1) with an optimal dimension in sensing the axial bio-magnetic signal, into a coil array. It should be understood that 7 is intended only as a non-limiting example, and that different numbers of coils are possible. Operating on Faraday's law, the extremely weak magnetic flux from the heart can be translated into voltage across all coils. Leveraging digital signal processing (DSP) (e.g., bandpass filter(s) and averaging over different coils, MCG can be retrieved in real-time. The study included in-vivo validation tests that confirm that the coil array can monitor real-time human MCG with clear QRS complex in non-shielded environment and when averaging over 4.5 minutes, clear P, T, and U waves can be identified. In-vivo repeatability tests confirm for intra- and inter-subjects, cardiac cycle detection accuracy (>99.13%) and averaged R-R interval accuracy (<5.8 ms) are highly comparable to the gold-standard ECG device while being robust to body movement. The example implementation can include a variety of improvements over conventional methods, including that it may not suffer from mapping limitations, low sensitivity, tissue attenuation (ECG sensor), high cost, bulkiness and shielding (SQUIDs), high temperature safety concern (AMs), extensive averaging time, and/or may not require an ECG signal. These and other advantages over conventional systems and methods are described throughout the present disclosure. Moreover, implementations of the present disclosure can leverage e-textile technology, and can optionally be seamlessly integrated into garments enabling constant cardiac sensing which help bringing cardiac monitoring to the patients at their own comfort.

Magnetocardiography (MCG) can be a non-invasive and non-contact means of capturing the magnetic fields radiated by the human heart. Clinical benefits of MCG compared to electrocardiograman include: (1) the ability to provide three-dimensional mapping of the heart [1A]; (2) high sensitivity toward tangential and vortex currents [2A]; and (3) clear and reliable detection of cardiac activity through thick tissue (e.g., monitoring of fetal cardiac activity) given that magnetic fields propagate relatively undisturbed though body tissues (tissue permeability ≈0) [3A]. MCG can, in various implementations, monitor coronary artery diseases in patients without persistent ECG features, identify early repolarization patterns to prevent ventricular fibrillation, provide more accurate prognosis of ventricular tachycardia, test rejection reaction post heart transplantation, and detect various related fetal cardiac conditions [4A-9A].

Technologies are available for capturing the extremely weak MCG signals (range of 50-100 pT) [4A]. Clinical practice mainly uses superconducting quantum interference devices (SQUIDs) [10A] that convert the cardiac magnetic flux into oscillating voltage via Josephson junctions in extremely low-noise environments. To achieve the desired performance, SQUIDs need to operate in a super conducting state (operating temperature of ˜4 K to ˜77 K) and in the presence of magnetic shielding. In turn, SQUIDs are bulky, non-portable, and expensive. Recent technology has introduced different types of atomic magnetometers (AMs), such as Optically Pumped Magnetometers (OPM) and Spin Exchange Relaxation-Free (SERF) magnetometers, to sense MCG signals. AMs achieve similar detection sensitivity as SQUIDs with much smaller size and without the need for cryogenics [11A]. Nevertheless, AMs are active devices that require alkali atoms to be heated to a high temperature (˜150° C.), raising safety concerns. In addition, most AMs can only operate in a near-zero magnetic field and their signal bandwidth is extremely limited [12A]. Very few OPMs (such as the QuSpin Total Field Magnetometer, QTFM) can operate in earth ambient noise, but at the cost of degraded noise performance [13A]. AM may not be able to record clear human MCG in the absence of shielding. [13A]. Efforts to overcome the key limitations of AM are limited, for example they may not include MCG detection in real time, or may not be passive devices [14A], [15A].

Implementations of the present disclosure include sensors where alternating magnetic flux from the heart interacts with induction coils placed upon the chest to induce voltages upon them. Through the theoretical optimization of the coil geometry and advanced digital signal processing (DSP), the final MCG signal can be retrieved in an earth ambient field [18A]. This sensor is comparable in size, weight, and sensitivity to a typical AM sensor (i.e., ˜ centimeter, ˜ grams, and ˜ pT/\sqrt{HZ}, respectively); fully passive without the need of any heated alkali atoms nor any type of cryogenic; and capable of recording emulated signals that mimic the human MCG activity.

The present study includes an example implementation of the present disclosure including a low-cost sensing system that (a) monitors the full spectrum of MCG activity with: i. clear R-peaks in real-time and ii. fairly close P, T, QRS, and U waves over ˜4.5 min of averaging, (b) enables stand-alone operation of the MCG sensor without the need for an accompanying ECG sensor, and (c) has been validated in vivo on human subjects. As used herein, “full spectrum” indicates the full-frequency spectrum information, and “clear R-peaks” are visually identified across the time-domain measurement.

To verify that the signal captured is indeed MCG and not chest wall vibration, the study conducted a proof-of-concept test with the MCG sensor placed at a distance away from the participant's chest. Comparing the example implementation to the state-of-the-art, the reported MCG sensor (1) exhibits very low operation cost (per use) and fabrication cost, unlike SQUIDs that cost USD ˜5000 just to be turned on [19A]; (2) does not require any accompanying structures to operate, unlike SQUIDs that require coolant to maintain an operating temperature of from ˜4 K to ˜77 K and unlike AMs that require heating/thermal isolation structures as well as shielding; (3) can detect the R-peaks of MCG in real-time serving as a self-sufficient device for subsequent averaging, unlike previous induction coils that require an accompanying ECG device to capture the R-peaks [14A]; and (4) is fully passive without any lasers or strong magnetic fields needing to be applied, unlike AMs and [15A].

The study included eleven (11) healthy volunteers between 22 and 35 years (M=26.4 years, SD=4.2 years) of age. The sample size of the recruited participants was selected to be comparable to other studies [20A-22A]. Demographic and physical characteristics data (age, gender, height, weight) were collected prior to conducting the experiments.

Of the 11 enrolled participants, 4 identified as female (36%) and the other 7 identified as male (64%). The participant sample consists of females aged between 22 and 35 (standard deviation, SD=5.6 years), height between 163 cm and 170 cm (SD=2.9 cm), weight between 48.5 kg and 66 kg (SD=7.2 kg), and Body Mass Index (BMI) between 18.3 kg/m² and 22.8 kg/m² (SD=1.9 kg/m²). The male participants were aged between 23 and 30 (SD=2.4 years), height between 165 cm and 192 cm (SD=8.4 cm), weight between 55 kg and 82 kg (SD=11.8 kg), and BMI between 19 kg/m² and 26.1 kg/m² (SD=2.9 kg/m²).

The recording system employed in this study is shown in FIG. 12A and can include MCG sensors 1220 and ECG sensors 1210, an Analog to Digital Converter (ADC) 1230, and a computer 1225 for storing and processing the data. It should be understood that the computer 1225 can include any or all of the features of the computing device 400 shown in FIG. 4. It should also be understood that the computer can include software and/or hardware configured to perform digital signal processing on the signals received from the MCG sensor 1220, ECG sensor 1210, and/or from the ADC 1230.

FIG. 12B illustrates a block diagram 1250 of the system 1200 shown in FIG. 12A. The block diagram 1250 includes the MCG sensor 1220, ECG sensor 1210, computer 1225, and ADC 1230 shown in FIG. 12A. The block diagram 1250 further illustrates a power supply 1236 that can optionally be used to power an amplifier 1232. The amplifier 1232 can be used to amplify signals from the MCG sensor 1220 before those signals are received by the ADC 1230. A battery 1234 can optionally be used to power the ECG sensor 1210. It should be understood that the battery 1234 and power supply 1236 are intended only as non-limiting examples, and that implementations of the present disclosure can by powered by any combination of power supplies 1236, batteries 1234, and any other power sources.

The ECG data were collected as the “gold-standard” electric equivalent of MCG with a goal to validate the performance of the MCG sensor. In real-world scenarios, cardiac activity can be ultimately monitored directly from the MCG sensor itself without the need for a concurrent ECG recording. The example implementation can optionally monitor cardiac activity without concurrent ECG recording, [17A].

The MCG sensor can be based on the operating principles described in Example 1. To achieve the target performance (i.e., real-time monitoring of the full MCG spectrum), the example implementation included a modified MCG sensor design to include 7 identical coils integrated into an array 8 cm in diameter. Each coil is of an optimal dimension in detecting the axial direction of the magnetic field, viz., Di (inner diameter)/D (outer diameter)=0.56 and 1 (length or height of the coil)/D=0.7182, as discussed in [16]. The exact coil parameters are selected as in [18A] to match the high sensitivity performance, viz., Di=9.3 mm, 1=12 mm, and D=16.6 mm. The coils are based on an optimized tightly winded air-core coil design [16] with design parameters being represented by the coil geometry (i.e., outer/inner diameter, wire diameter, and coil height/length). Applying the tightly wound coil model, the number of turns is calculated to be ˜1095. However, the exact number of coil turns are not provided to the coil manufacturer and the actual number of turns may slightly vary per the employed winding technique and fill factor. The total number of 7 coils is determined by first using a single-coil sensor to collect human MCG data as a function of time. With that, 7 cardiac cycles are identified as the minimum number of cycles needed to be averaged to see a clear MCG signal. This is due to the reduced level of uncorrelated noise [16A, 18A].

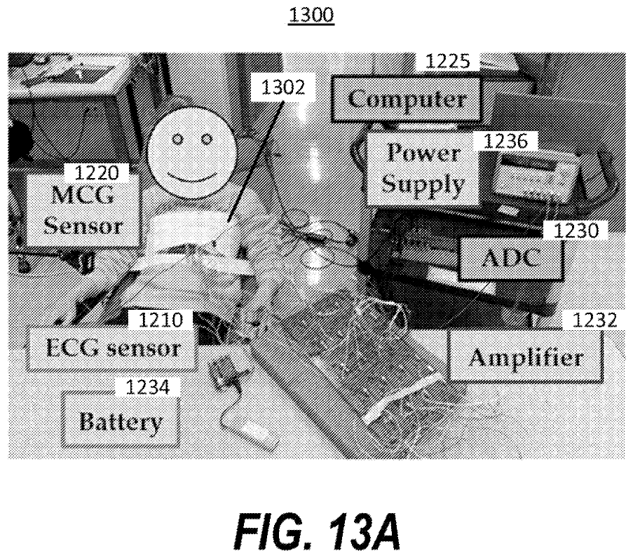

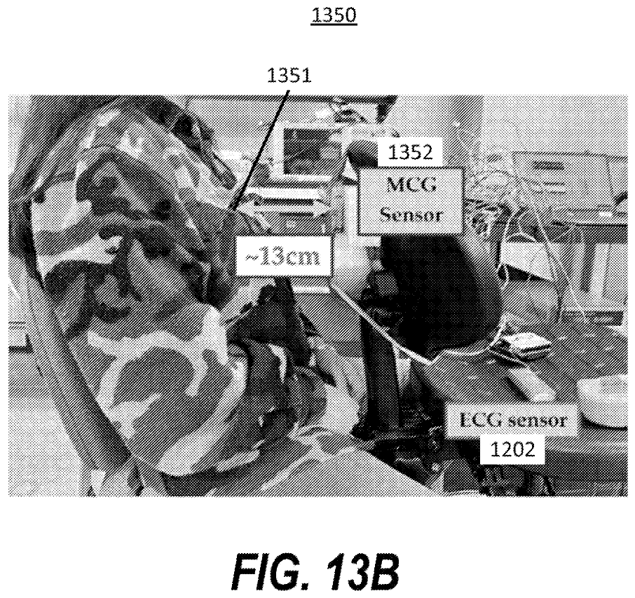

Implementations of the present disclosure can monitor the MCG data in real-time, instead of averaging over cardiac cycles. The study included averaging over 7 different coil recordings that capture data at the same time. Since coils can be small in size and can optionally be placed in close proximity to each other, they can often capture the same signal. Referring to FIG. 12A, 1 coil can be placed in the center of the fixture and the remaining 6 can be placed evenly surrounding it. The MCG sensor can be covered with very thin and electromagnetically transparent green tape to keep the coils in place. It can then secured on to an elastic chest belt 1302, as shown in FIG. 13A.

Compared to the example implementation described in Example 1, the coil sensor of the present example is slightly larger with its geometry better fitting the calculated ideal ratio of dimensions. An example coil 1246 is shown in FIG. 12A. The example coil's outer radius (D) changed from 15 mm (reported in [16]) to 16.6 mm, and the coil's length (l) changed from 11 mm (reported in [16A]) to 12 mm. The coil's inner diameter (Di) and wire diameter (d) were kept the same as in [16A]. Seven total coils 1246 are used in the example implementation, were arranged in a flower-like arrangement supported by a 3D-printed fixture with a total diameter of 8 cm. The fixture includes seven tightly fitted slots, with one for each of the coils. Six slots 1244 are centered along the edges of a hexagon 1242 and the seventh slot 1245 is placed in the center of the hexagon 1242, as shown in FIG. 12A. The distance between the center coil and each side coil is 22 mm. This design is selected to maximize the number of coils across a relatively small circular area as needed for both localized MCG detection and averaged real-time MCG detection. It should be understood that the shape and size of the coils, the number of coils, and the placement of the coils relative to one another can be changed for different applications and different systems, and that the shape and size of the coils, the number of coils, and the placement of the coils relative to one another given herein are intended only as non-limiting examples. For example, in an alternative implementation, the hexagon 1242 could be replaced with any other shape, with any number of slots 1244 for coils.

Seven (7) low noise amplifiers (gain=1000) can connect to each of the coils. It should be understood that different numbers or combinations of low-noise amplifiers, and/or different gains can be used. The low-noise amplifiers can be fabricated on a single circuit board and placed far away from the sensors to reduce the vibration and electronic noise. A detailed circuit design is shown in [16A], where the input network, voltage regulator, low noise instrumental amplifier (INA217), and reference pin offset correction (OPA2277) are the building blocks for the employed amplifier. Optionally, all signal processing is performed digitally to reduce the additional noise. A power supply can be constantly powering the amplifier board with +10 V. The noise performance (noise spectrum density) of the sensing system (one coil sensor and one amplifier) can be identical to that of the non-shieled environment [18A].

Implementations of the present disclosure can use components that can be simple and cheap to manufacture. For example, the overall cost can be very low for the proposed sensing system. Each coil sensor costs less than USD 1 to manufacture and there is no additional cost for shielding/any types of coolant/any accompanying structures. The fixture holding the sensor is based on standard 3D-printing approaches, while the circuit board includes low-cost components including one INA 217 (USD 6), one OPA 2277 (USD 5), voltage regulators (UA78L05ACDRG4 and MC79L05ACDG, USD 1 total), and additional resistors/capacitors/connectors. For one amplifier board, the total cost is USD ˜38 and the price can be significantly reduced when ordered in bulk quantities.

The ECG signals can be recorded using a 3-lead ECG sensor (e.g., the “e-Health sensor shield” sold under the trademark Arduino [23A]) powered by a battery. Optionally, both analog signals (ECG and MCG) are converted to digital ones using a 4-channel, 24-bits National Instrument ADC (NI9239) that samples at 5 kHz through a National Instruments Lab VIEW interface. Since 8 channels are needed to concurrently record ECG and MCG data (1 for ECG and 7 for each of the MCG sensor coils), 2 identical ADCs (NI9239) are combined in a CompactDAQ chassis (NI cDAQ-9189) and perform simultaneous multi-channel data acquisition.

The study experimental setup 1300 is shown in FIG. 13A. The tests in the present study were performed in a regular Radio-Frequency (RF) laboratory environment with a decent level of electronic noise and no shielding.

In the study, the MCG chest belt was wrapped tightly around the participant's chest with the sensor array located in front of the chest and sitting slightly to the left of the breastbone 1202 shown in FIG. 12A). This configuration minimizes noise due to body movement and maximizes the magnetic cardiac signal strength as captured via the MCG sensor 1220. Both the amplifier 1232 board and the power supply 1236 are placed far away from the participant to reduce electronic noise (on a workbench in the front of the participant and a cart placed on the side, respectively, as shown in FIG. 13A). FIG. 12A illustrates example locations for ECG electrodes and MCG electrodes. Three foam-based disposable ECG silver/silver chloride (Ag/AgCl) solid gel electrode pads are adhered to the left-hand wrist 1204, right-hand wrist 1206, and the left-side lower edge rib cage 1208 to record the ECG. The neutral, positive, and negative leads from the ECG sensor 1210 are connected to the three pads, respectively. Here, an “Arduino-based” ECG is used as the “gold standard” for comparison between the MCG and ECG signals. The “Arduino-based” ECG was used for its low cost and off-the-shelf availability. The ADC 1230 that simultaneously recorded the ECG and MCG signals, as well as the laptop computer 1225 that stored the data were placed on a cart next to the participant.

The present study included two studies performed back-to-back, each lasting ˜4.5 min. The participant was instructed to lie back on a zero-gravity chair with no body movement. Here, the study purposely eliminated body movement to reduce noise to the largest possible extent. As used herein, this is referred to as a “no movement study”. Next, the participant is instructed to sit in an upright position (without his/her back lying upon the chair) and tap upon a cell phone with RF features turned off. This setup is used to increase the noise due to motion and better mimic a real-world scenario. Despite operating the phone in “airplane mode”, the setup is not considered as “shielded”. This is because the RF signal from the phone lies outside the frequency range of the MCG signal. Even if it were present, this RF noise could easily be filtered out through DSP. The reason for operating the phone in “airplane mode” is to prevent any sort of distraction on behalf of the participants rather than shielding the experimental setup in any way. As used herein, this study is referred to as a “minimal movement study”.

To derive the MCG signal, the study employed two DSP methods: (1) bandpass filtering, and (2) averaging over different coils [18A]. Compared to DSP [16A], the present study eliminated the need for any accompanying trigger signal to identify the repeating cardiac cycles. In alternative implementations, to reduce noise, the MCG can be recorded for a period of time together with a trigger signal (i.e., ECG). Using the R-peaks identified in the ECG, each cardiac cycle (e.g., 1 s window containing one R-peak) can be subsequently identified in the raw MCG signal. These identified cardiac cycles in the MCG can then be averaged to produce the final clear MCG signal. In the implementation described with reference to the present example,, instead of ECG, averaging the MCG signal itself over seven (7) coils can produce a clear R-peak that can be used as a self-trigger to identify repeating cardiac cycles. In the example implementation, 7 was identified as the minimum number of cycles needed to be averaged to see a clear MCG R-peak. Again, it should be noted that 7 is intended only as an example, and that any number of coils can be used in various implementations of the present disclosure. Specifically, the raw MCG signals from each of the 7 coils, viz., MCG raw1 . . . MCG_raw7, first pass through a bandpass filter to reject the frequencies outside of the target frequency range (4-30 Hz), generating the signals MCGf_1 . . . . MCGf_7. The bandpass filter can be implemented using the default MATLAB built-in “bandpass” function. It is designed as a minimal-order Finite Impulse Response (FIR) filter when the input signal (viz., MCG_raw1 . . . MCG_raw7) is long enough, or a minimal-order Infinite Impulse Response (IIR) filter otherwise. In the present case, all bandpass filters are FIR with 60 dB stop band attenuation and 0.1 pass band ripple. The delay of the filtered signal is compensated. The filtered signals (MCGf_1 . . . . MCGf_7) are then averaged based on the winding direction, as discussed next, to derive the final real-time MCG signal (MCG_{final}).

Optionally, the winding direction of each of the coils can be considered in the averaging process. To determine the correct direction, a high-magnitude pre-recorded ECG signal can be fed into a circular loop wire using a function generator and the converted magnetic signal can be picked up by the proposed MCG sensing system. In some implementations, the raw signals captured by all 7 coils are large enough, such that the direction of winding for each coil can be determined without DSP. In the study of the example implementation, the calibration was performed using a 4 cm radius single-turn circular loop wire fed by a function generator at 10 Vpk-pk using a pre-recorded human ECG signal (the input impedance of the function generator being 500). All 7 coils were fixed onto the flower-shaped structure described herein to secure them in place and the whole structure is placed 15 mm away from the circular loop wire picking up the perpendicular magnetic field. With such a strong original signal, the signal that is being picked up can be clearly seen after averaging over repeating cycles (per [18]). This direction can be calibrated during the coil averaging process by adding a negative sign to those signals with an opposite direction. With that, in the present case, the MCG_{final} is calculated as:

$${MCG}_{final} = \frac{{\sum }_{i = 1}^{3} MCGf\_i - MCGf\_ 4 + MCGf 5 - {\sum }_{n = 6}^{7} MCGf\_n}{7}$$

To evaluate the performance of the MCG sensing system, the study carried out two tests:
(1) Validation test. This test serves to validate (a) the feasibility of real-time MCG detection, and (b) the extraction of detailed MCG features through averaging over multiple cardiac cycles (or, equivalently, over a period). For this proof-of-concept validation step, the study used data collected from the “no movement study” of a single participant (28 years old female; height 1.63 m, weight 48.5 kg, and BMI 18.3 kg/m²). Specifically: (a) The real-time MCG detection is validated by confirming the presence of synchronized peaks in the ECG and MCG_{final} signals. Here, synchronization refers to the ability of the major spikes of ECG and MCG_{final} (known as R-peaks) to identify the same number of cardiac cycles across the same period. The exact alignment of the R-peaks between the ECG and MCG_{final} signals may not be anticipated in the time-domain as MCG is actually the derivative of ECG.
(b) The MCG signals are averaged throughout the test duration, viz., across 4.5 min, to explore whether and which detailed features can be detected (such as P, T, and U waves). The process of averaging over repeating cycles has been described in [18A] as a means of improving the MCG detection sensitivity. As described herein, concurrent ECG signal can optionally serve as a trigger and identify the MCG cycles. In the present example, the ECG trigger may not be required as the R-peaks can be detected in real-time using a stand-alone MCG sensor configuration. Hence, the example implementation can readily identify repeating cardiac cycles using the R-peak in the MCG_{final} signal itself. Averaging over the entire test duration (˜4.5 min) is by no means limiting; shorter or longer averaging durations can be explored to derive the diverse features of the MCG signal.
(2) The study further included repeatability tests. These tests entail intra-subject and inter-subject repeatability tests and serve to evaluate (a) the sensor's detection accuracy vs. gold-standard ECG and (b) its tolerance to body movements. Specifically: (a) Data recorded during the “no movement study” are used to analyze the sensor's detection accuracy in terms of two different metrics, namely, QRS detection accuracy and average R-R interval accuracy. The former compares the number of visible QRS waves in the MCG signal (MCG_{QRS}) as opposed to the number of visible QRS waves in the gold-standard ECG signal (ECG_{QRS}) over the entire recording period and is defined as:

$${Accuracy}_{QRS} = \frac{{MCG}_{QRS}}{{ECG}_{QRS}} \times 100 \%$$

To determine the average R-R interval accuracy, the average R-R interval is calculated for only those sections of time with 100% QRS detection accuracy (i.e., the same number of R-peaks detected in both the MCG and ECG). This is performed to eliminate the chance of missing cycles, which can lead to erroneous R-R interval calculations. Assuming that n number of R-peaks are detected within this timeframe and that Rn represents the time when the corresponding R peak is identified, the average R-R interval is calculated as the sum of the times between all the two neighboring R-peaks divided by the number of R-R intervals.

$$Average R - R interval = \frac{{\sum }_{i = 1}^{n - 1} {R}_{i + 1} - {R}_{i}}{n - 1}$$

Average R-R intervals are calculated for both MCG and ECG, viz., MCG_{R-R} and ECG_{R-R}, and the difference between the two is used as a second metric of accuracy for the MCG sensor:

$${Accuracy}_{R - R} = \vert {MCG}_{R - R} - {ECG}_{R - R} \vert$$

The MCG sensor tolerance to movement can be analyzed by comparing the data for the two study scenarios, i.e., “no movement study” and “minimal movement study”, throughout the test duration. The root mean square (RMS) of the isoelectric region and the minimum averaging time (or, equivalently, the minimum number of cardiac cycles) needed to identify the P and T waves are used to quantify the effect of the participants movement. Here, the isoelectric region, viz., the baseline region, is defined as the MCG_{final} signal having excluded all visible QRS waves. Since motion artifacts occur within the spectral content of the original MCG signal [24,25], excluding the signal itself from the source signal (where signal, motion artifacts, and other sources of noise are combined), provides a signal that better represents the participants' movement. Here, other sources of noise are assumed to be consistent across the two studies (i.e., “no movement study” and “minimal movement study”) as the data are collected one after another in almost the same time and environment. The QRS waves are excluded by deleting a 100 ms region across each detected R-peak, with 100 ms chosen as the widest QRS duration in healthy adults (per [26], QRS duration varies between 80 and 100 ms). Among standard measures of noise, viz., (1) root mean square (RMS) of isoelectric region; (2) ratio of R-peak to noise in the isoelectric region; (3) crest factor; (4) ratio between in-band and out-of-band power; and (5) power in the residual after filtering [27], the RMS of the isoelectric region is selected as the most accurate and direct means to represent the movement component of the noise. Additionally, (2) and (3) introduce the magnitude of the MCG signal into the equation, which complicate the process, while, for (4) and (5), the key component of the noise measured is the powerline noise (60 Hz), which lies outside the frequency of interest.

Finally, similar to the DSP procedure described in Section 2.5 (1) (b), cycle averaging is added to detect the detailed MCG features (i.e., P and T waves). A hypothesis is that, with higher values of movement noise, the P and T waves will be harder to detect, leading to longer values of minimum time (or, equivalently, an increased number of cardiac cycles) needed for averaging. In turn, this minimum averaging time (or, equivalently, minimum number of averaging cardiac cycles) is used herein as a parameter to quantify the noise. It is determined by gradually reducing the length of the MCG_{final} vector used for cycle averaging until the P and T waves cannot be visually identified. The minimum length of MCG_{final} that can retrieve visible P and T waves (MCG_{final}_min) is used to calculate the
‘timeₘᵢₙ’ and ‘cyclesₘᵢₙ’. Here, ‘timeₘᵢₙ’ is calculated as the minimal length (MCG_{final}_min)
divided by the sampling rate of the ADC (Fs) and ‘cycles’ is defined as the number of R-peaks within the selected length.

$${time}_{min} = \frac{{MCG}_{final \_ min}}{Fs}$$

The study further included MCG verification (verifying that the signal is MCG, and not based on chest vibrations). This can be used to confirm that the resulting signals are not based on chest vibrations like seismocardiography, as opposed to MCG. To verify that the signal is indeed MCG, the study modified the previous setup so that the 7-coil sensor is placed a few centimeters away from the subject's chest wall, i.e., none of the coils are touching the body. If the recorded signal is indeed MCG, it should be visible without any skin contact (though the increased distance from the chest may necessitate averaging over some extent of repeating cardiac cycles).

For this verification test, a 28-year-old female (height 1.63 m, weight 48.5 kg, and BMI 18.3 kg/m²) was recruited. Since the test was designed to confirm the MCG detection, only one participant is recruited as a proof of concept. The experimental setup 1350 is shown in FIG. 13B, with the distance between the chest wall 1351 and the 7-coil array 1352 being ˜13 cm. The materials surrounding this experimental setup (including the chair, table, etc.) are made of plastic, fabric, or foam, all of which have a relative permeability of close to 1. All the coils were pre-calibrated so that the corresponding winding is represented in the DSP, per Equation (1). The coils are recording continuously, along with the 3-lead ECG system described in Section 2.3. In this case, ECG is used as the gating signal to identify the repeating cardiac cycles for averaging.

Optionally, implementations of the present disclosure can use simple DSP. A non-limiting example of simple DSP only utilizes three methods, namely, bandpass filtering, averaging over repeating cycles, and averaging over multiple coils, as discussed in [16]. Taking a step further, the time width of the QRS acquired during the MCG validation test is further evaluated to confirm whether the measured waveform is indeed MCG. Here, the QRS is measured manually in the final averaged signal. To compensate for the signal degradation due to the additional 13 cm distance, data are recorded for ˜20 min, so that the study had enough cycles for averaging.

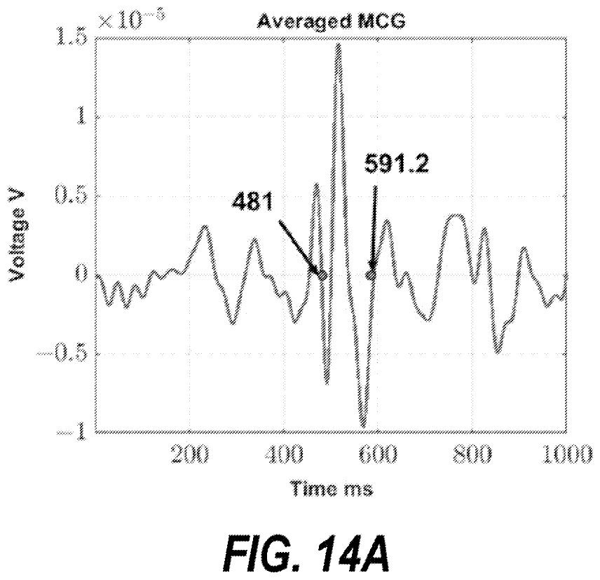

FIG. 14A shows the processed signal recorded 13 cm away from the chest. Expectedly, given the increased distance between the chest and the sensor, the MCG signal cannot be seen in real-time. Specifically, to generate the signal of FIG. 14A, ˜1 min out of the ˜20 min recording is used (i.e., 75 cardiac cycles per coil). The QRS in the averaged MCG is manually measured to be 110.2 ms, which is within the normal range when compared to the results reported in [28,29]. Since this experiment of the study is intended to confirm MCG detection, the setup is by no means optimized. Notably, the cardiac activity is detected by the sensor, confirming that the captured signal is indeed MCG.

FIG. 14B shows example MCG (solid line) and ECG (dashed line) voltage data recordings in real time. The x-axis represents the time stamp. Though the study starts at 0 min and ends at 4.5 min, FIG. 14B only shows data from 2.26 min to 2.39 min as an example. As seen, all R-peaks in the MCG can be clearly identified, and each peak is matched with a corresponding one in the gold-standard ECG. A similar correlation is found throughout the entire 4.5 min of the recording. This trend confirms the hypothesis that real-time MCG can indeed be captured with R-peaks that are clear in each cardiac cycle.

FIG. 14C shows the averaged MCG over the entire study duration (i.e., ˜4.5 min) which, in this particular recording, contains 313 cardiac cycles. The study identified the cardiac cycles using the R-peaks of the MCG signal (see FIG. 14B) and cut windows of 500 ms in duration in each side of the R-peak. In particular, the R-peaks are identified using the Matlab function “findpeak” with a defined minimal distance between the two peaks and defined minimal height of the peaks. In addition to the main QRS spikes, the P, T, and U waves can all be fairly closely identified in FIG. 14C, confirming that, with minimal averaging (4.5 min), detailed MCG features can be extracted.

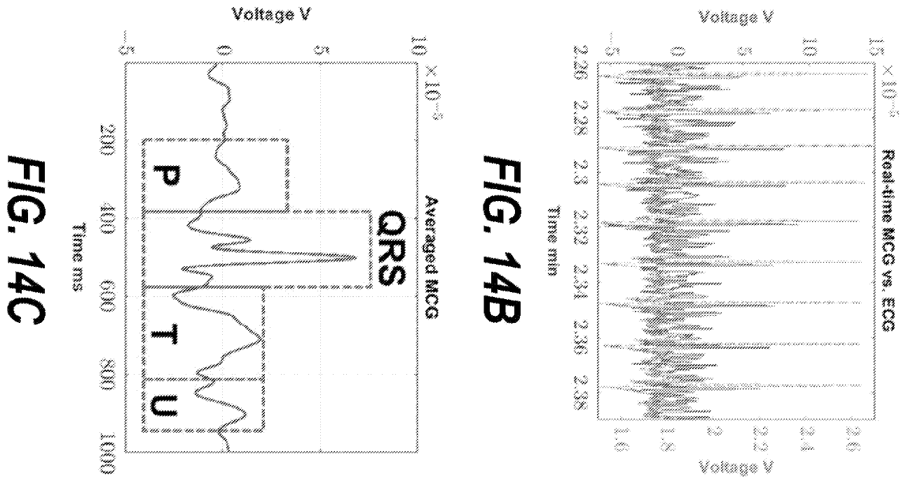

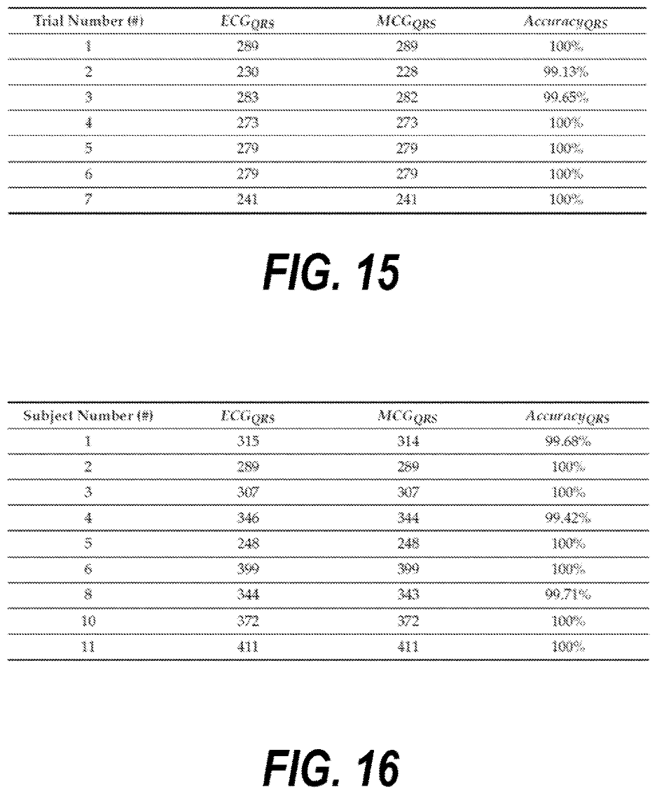

The intra- and inter-subject QRS detection accuracy can be calculated using Equation (2) and listed in FIG. 15 and FIG. 16, respectively. In the former case, a single participant (24 years old male; height 1.70 m, weight 55 kg, and BMI 19 kg/m²) repeats the “no movement study” seven different times. The QRS accuracy values (Accuracy_{QRS}) are calculated using the entire data recording of ˜4.5 min. The QRS/R-peak detection is again realized using the “findpeak” function in Matlab with a defined minimal distance between the two peaks and defined minimal heights of the peaks. As seen, clear MCG QRS waves are retrieved from all intra-subject trials and from 9 out of the 11 participants. In particular, subjects #7 and #9 are both female with thicker breast tissue and higher BMI (22.8 kg/m² and 22.2 kg/m², respectively), leading to increased distance between the signal source (heart) and the MCG sensor and, hence, lower signal quality. To put this in perspective, the two females that are included in this study have a BMI of 18.3 kg/m² and 20.3 kg/m², respectively. The study excluded these two participants from subsequent data analysis, yet future advances in the hardware and signal processing will aim to improve the sensor performance. Referring to FIG. 15 and FIG. 16, the QRS detection accuracy among all intra- and inter-subject trials is >99.13%, with 5/7 trials (intra-subject) and 6/9 subjects (inter-subject) achieving 100% detection accuracy as compared to the gold-standard ECG.

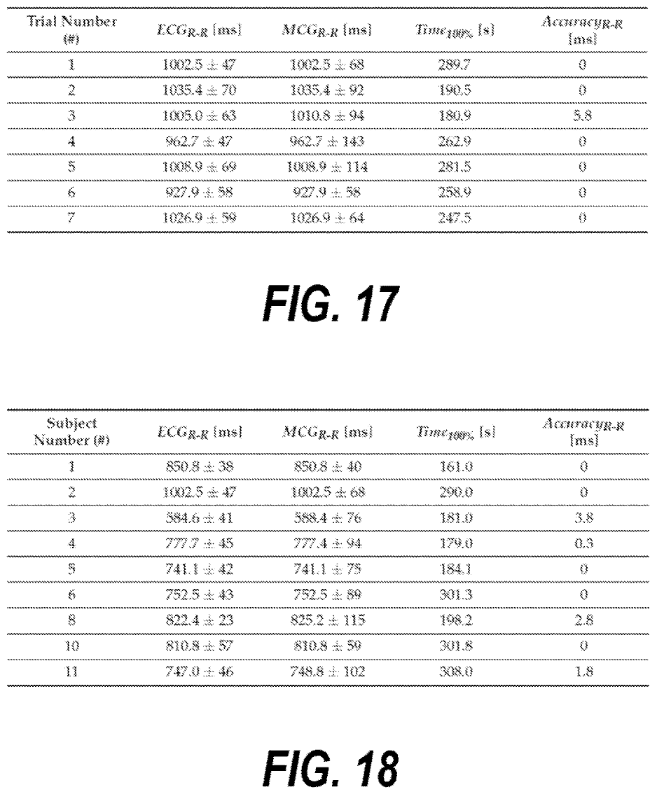

The averaged R-R intervals ((SD) and the R-R accuracy are calculated using Equations (3) and (4) and are summarized in FIGS. 17 and 18 for the intra- and inter-subject tests, respectively. Here, Time_{100%} refers to the continuous time duration with a 100% QRS accuracy rate. Among all, the difference between the MCG_{R-R} and ECG_{R-R} is always <5.8 ms, with 6/7 trials (intra-subject) and 5/9 subjects (inter-subject) exhibiting 100% identical intervals. Comparing FIG. 17 and FIG. 18, the number of cases with Accuracy_{R-R}=0 are slightly higher for the intra-subject tests. On the other hand, compared within FIGS. 17 and 18, the standard deviations for MCG_{R-R} are slightly higher than those for ECG_{R-R}. Nevertheless, for all intra- and inter-subject tests, the discrepancy between the averaged R-R intervals and the standard deviation calculated in MCG and ECG are negligible.

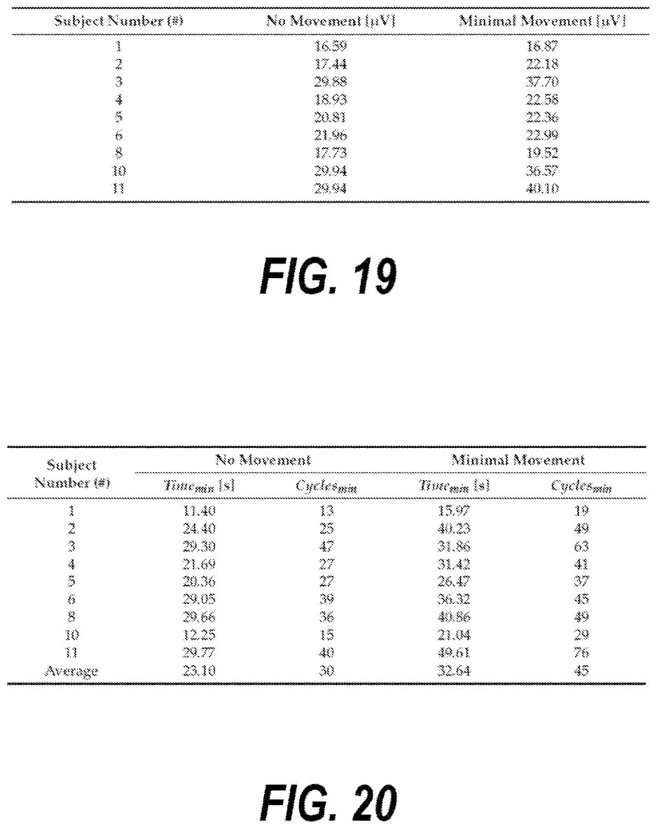

FIG. 19 shows the RMS of the isoelectric region of the MCG in two scenarios, viz., no movement and minimal movement. As expected, higher RMS values are observed in all minimal movement studies due to the additional motion artifacts that are introduced into the recording. Here, data from the entire test duration (˜4.5 min) are used to calculate the RMS value, and the 100 ms regions surrounding the R-peaks are deleted for those R-peaks that are detectable in Matlab. Since the MCG sensor has already proven to exhibit a QRS accuracy of >99.13% (see FIG. 15), only very few regions are left out. For each participant, the RMS values of the isoelectric region are different due to each test being conducted on different days with different environmental noise. Nevertheless, all values are in a similar level of signal strength (˜10⁻⁵ V).

FIG. 20 shows the minimum time and minimum number of cycles needed to identify the detailed MCG features, specifically the fairly closed P, T waves, for each of the “no movement” and “minimal movement” studies. As would be expected, for all nine participants, a longer averaging time is needed to identify the P, T waves when retrieving MCG under minimal body movement. On average, the P, T waves can be identified by averaging over 23.1 s/30 cardiac cycles during the “no movement study” and 32.64 s/45 cardiac cycles for the “minimal movement study”. In all cases, the detailed MCG features can be extracted in less than 50 s/76 cardiac cycles. Since different participants have different heart rates, the number of cardiac cycles used for averaging is a better representation of the DSP process. For example, the participants with faster heart rates have an advantage in terms of the minimum time needed for average as, within the same period of time, more cardiac cycles are present.

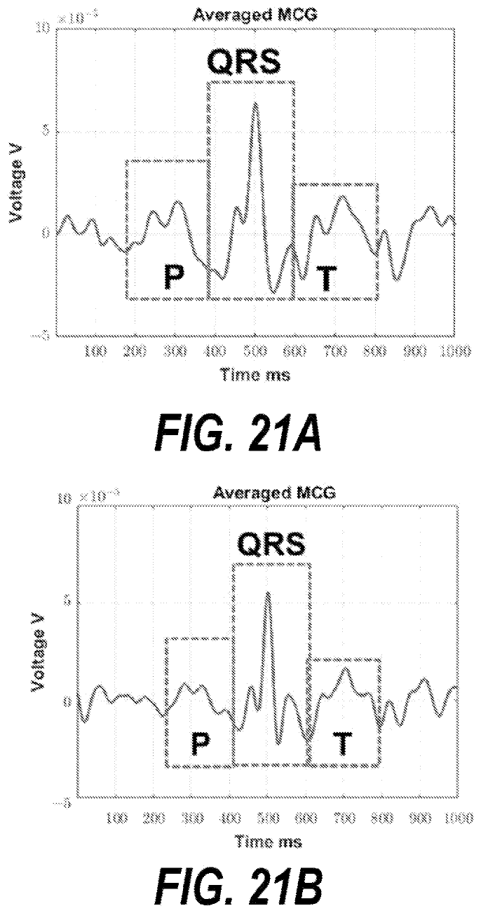

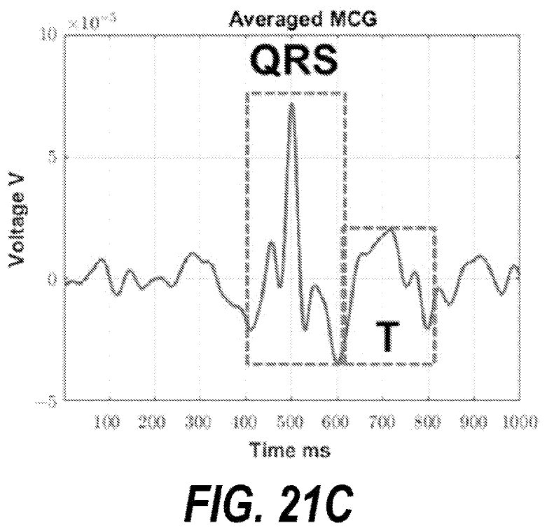

To better understand the impact of movement, FIG. 21A shows the processed MCG plot for one of the participants using 11.40 s/13 cardiac cycles of averaging time for the “no movement study” (identified in FIG. 20 as the minimum time needed for averaging when no movement is present in the example implementation). FIG. 21B shows the processed MCG plot using 15.97 s/19 cardiac cycles of averaging time for the “minimal movement study” (identified in FIG. 20 as the minimum time needed for averaging when minimal movement is present), and FIG. 21C shows the processed MCG plot averaging over 13 cardiac cycles (same number of minimum cycles used for “no movement study”) for the “minimal movement study”. As seen, the P, T waves are quite visible in FIG. 21A and FIG. 21B as most of the uncorrelated noise (i.e., motion artifacts) is eliminated with the increased averaging time in the second case. By contrast, the P wave is not visible in FIG. 21C as the reduced number of averaging cycles is not sufficient to fully remove the motion artifacts. Here, the P wave is of a similar amplitude to that of the other waves in the isoelectric region. By contrast, the T wave is somewhat visible due to its higher amplitude. To sum up, regardless of the recording situation (e.g., “no movement” vs. “minimal movement”), the MCG signal can be retrieved with modifications in the recording time and the signal level for the same participant should be the same.

The example implementation of a real-time unshielded MCG detection system provides a solution for low-cost and continuous MCG monitoring. The example implementation can therefore be useful in contexts outside the hospital setting. The results of the study further indicate the use of implementations of the present disclosure for (1) R-peak monitoring in real-time (e.g., to retrieve heart rate variability), (2) detailed MCG feature detection with ˜4.5 min of averaging, and (3) MCG tracking with no and minimal movement.

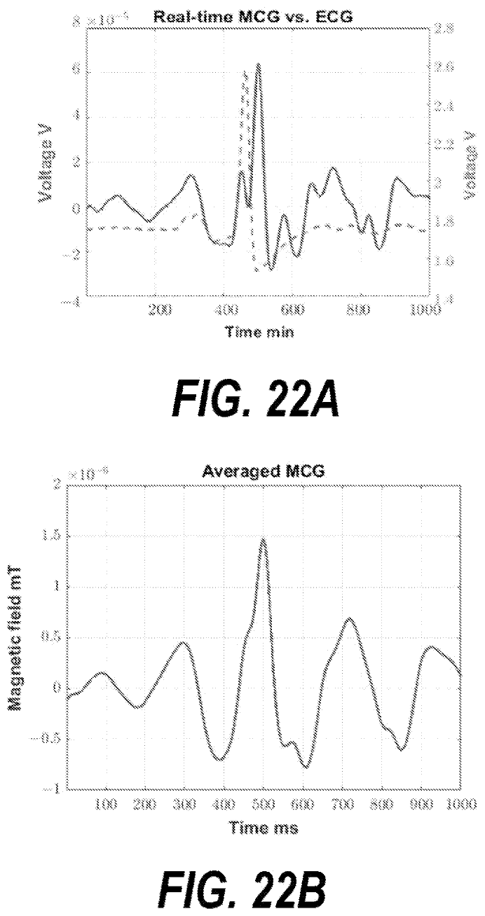

To confirm that detailed MCG features are valid features and not artifacts, the study superimposed the cycle-averaged ECG (dashed line) on top of the cycle-averaged MCG (solid line) using the data obtained in the validation test (FIG. 22A). Here, real-time R peaks (identified in MCG_{final}) are used as THE trigger to identify repeating cardiac cycles for both ECG and MCG. As seen, only P-waves can be identified in the final cycle-averaged ECG signal using a low-cost off-the-shelf device. The location of this P-wave aligns with that of the MCG signal though, of course, and perfect alignment is not anticipated (note that the voltage induced is proportional to changes in the magnetic flux over time). It should be understood that additional waves may be seen in implementations of the present disclosure using different devices.

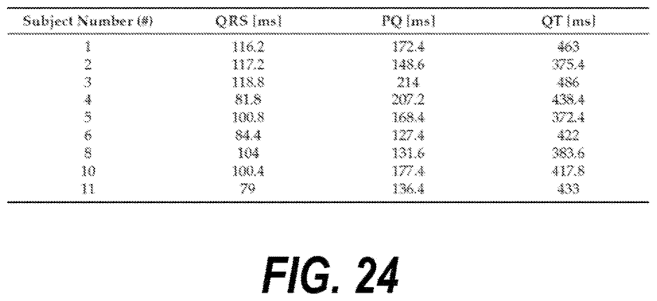

Additional data regarding the time widths of QRS, PQ, and QT for all recruited subjects during the minimal movement study are included in FIG. 24 to further validate that the detected waveforms in all cases are indeed MCG. Implementations of the present disclosure can include more extensive signal averaging and more advanced signal processing will further clear up the MCG signal, while clinical-grade ECG equipment can demonstrate the correlation for other detailed features of the cardiac waveform (such as T and U waves).

The main goal of the present study is to visualize the R-peaks of MCG in real-time so they can be subsequently used as a self-triggering signal by implementations of the present disclosure. As such, the study selected the 4-30 Hz frequency components of MCG as they contain most of the key features, particularly the R-peak. These detected R-peaks can then be used as a self-trigger for the MCG waveform filtered across a wider frequency range per application needs (e.g., 0.03-125 Hz [30] to identify ischemic ST elevation). Comparing FIG. 14A with FIG. 14C, the averaged MCG signal is obtained on the same subject with some distance (FIG. 14A) and without any distance (FIG. 14C) between the sensor and the chest wall. As seen, the R peaks can be detected in both cases, but with a reduced signal strength level when recording at a certain distance away. This is expected since the magnetic field strength produced by the heart is gradually degrading as the distance between the coil sensor and heart increases. In turn, detailed MCG features can barely be identified in FIG. 14A.

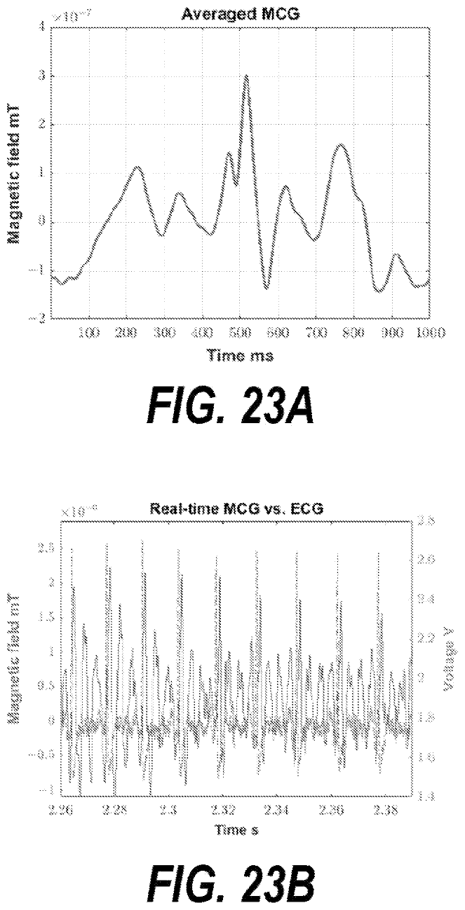

In the present example, all MCG signals are represented in terms of voltage as a more straightforward representation, given that the cardiac magnetic flux is translated into voltage by the coil sensors. This voltage can be translated back to the magnetic flux (Tesla) by using a Helmholtz coil and providing a known uniform magnetic field in a shielded environment. The calibration can then be performed with the known magnetic field and the corresponding recorded voltage. When converting to magnetic field, the environmental and instrumental noise may complicate the process, making the converted results more prone to error. For instance, the uniform magnetic field produced by the Helmholtz coil can be altered by the additional noise present in the shielded environment. That is, the translation relies greatly on the effectiveness of the shielding. Since the study includes extremely low field strengths, even a small alteration in the background field can greatly impact the final results. FIG. 22B shows an example plot with the signal converted from voltage to magnetic field using the data obtained in the validation test (FIG. 14A), while the rest of the plots have been converted to a magnetic field and are included in FIG. 23A. FIG. 23B illustrates a validation test plot showing real-time MCG vs. ECG in earth ambient noise, according to an example implementation of the present disclosure.

The converted signal strength (R-peak amplitude) in FIG. 22B is in the range of 10⁻⁹ T (˜1.5×10⁻⁹ T), which is bigger than the MCG amplitude typically expected from state-of-the-art sensors (˜10⁻¹⁰ T). This may be due to the distance between the heart and the recording sensor being smaller when compared to the typical recording setup using SQUIDs (>10 cm) [31A]. In the study, the sensor is placed directly on top of the chest wall. Studies of sensors recording at a shorter distance away from the heart have also shown similar results (i.e., R-peak values of ˜10⁻⁹ T) [32,33]. As expected, the converted plot magnifies the lower frequency components and reduces the higher frequency components. Further, to perform such conversion (i.e., from voltage to magnetic field), Fast Fourier Transform (FFT) and Inverse Fast Fourier Transform (IFFT) need to be performed, which introduce additional error. This error becomes significant when dealing with weak signals. Along these lines, the manuscript shows the originally captured units (Volts) as the most representative means to represent the recorded MCG signal. As such, when voltage is used as the direct means of measurement, such conversion is not typically adapted in the literature [14A, 34A-36A].

As a comparison, current clinical grade SQUID systems can achieve a sensitivity of ˜10-100 fT/\sqrt{Hz} around 10 Hz in shielded environments [10A-13A], whereas the example implementation of the system can achieve ˜30 pT/\sqrt{Hz} around the same frequency, yet in non-shielded environments. As such, some detailed MCG features, such as P and T waves are not as clear as those presented in SQUID-recorded MCG. The present disclosure contemplates the use of bigger coils, ferrite cores, partial shielding, and/or coolant approaches used in the sensor to further improve its sensitivity. When analyzing the effects of movement, as a proof-of-concept, the RMS of the isoelectric region is evaluated. This may not be the optimal method for quantifying the noise introduced by the movement, as some of the MCG waveform information overlaps with the isoelectric region. Implementations of the present disclosure can include better parameters to quantify the impact of movement.

Comparing the intra- and inter-subject test results, the overall performance is better for the intra-subject tests, viz., the number of cases in which A_{ccuracy}_{_{R-R}}=0 is higher as per FIG. 17 and FIG. 18. This can be explained by the fact that, as the participant performs multiple trials, they become more familiarized with the procedure, reducing motion artifacts in the recordings and leading to more accurate R-R interval values. Concurrently, for each subject and trials, MCG_{R-R} tends to vary in values more than ECG_{R-R}, viz., standard deviations for MCG_{R-R} are slightly higher than ECG_{R-R}. This may be due to the MCG signal, and particularly the MCG-derived R-peaks, being slightly distorted by noise. It is also worth noting that two female participants that have higher BMI (22.8 kg/m² and 22.2 kg/m², respectively) are excluded for all inter-subject studies due to low signal quality. Here, the higher BMI is not the sole reason for poor signal quality, but rather the key association lies in the distance between the heart and the sensor. For example, for male participants, even with higher BMI (subject #4 and subject #8, male, both having a BMI of 26.1 kg/m²), the MCG signal can still be retrieved. For this reason, the participants were divided not only based on BMI but also on gender. Considering this is a prototype recording system and a proof-of-concept experimental setup, more robust system hardware and better experimental setups will be explored in the future.

The example implementation of the present disclosure includes a coil array that can detect the full frequency spectrum of a human MCG with visible R-peaks in real-time without any shielding. The example implementation can further include a coil array capable of detecting clear QRS detailed MCG features (P, T, and U waves) with ˜4.5 min of averaging in non-shielded environments, without the need for any accompanying device to identify the cardiac cycles. The performance of the coil array was evaluated within and across subjects in terms of detection accuracy and tolerance to body movement. The in vivo experiments on human subjects confirmed that the detection accuracy for all intra- and inter-subject tests is >99.13%. The discrepancy in the averaged R-R intervals vs. gold-standard ECG is always <5.8 ms. When no movement is present, 23.1 s or 30 cardiac cycles are the minimal averaged time/cycles needed to identify the closed P, T waves. For minimal movement, these increased to 32.64 s and 45 cardiac cycles.

The reported sensor enables portable and low-cost MCG detection in real-time and non-shielded environments, showing promise for cardiac monitoring in the pre-clinical environment. Implementations of the present disclosure can leverage e-textile technology, to incorporate the systems and devices described herein into garments, enabling constant cardiac sensing.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### REFERENCES

The following patents, applications, and publications, as listed below and throughout this document, describes various application and systems that could be used in combination the exemplary system and are hereby incorporated by reference in their entirety herein.
[1] Peterson, L.; & Peterson, M. J. Short-term retention of individual verbal items. Journal of experimental psychology 1595, 58 (3), 193.
[2] Rozado, D.; Dunser, A. Combining EEG with pupillometry to improve cognitive workload detection. Computer 2015, 48 (10), 425-438.
[3] Mohammad, AA.; Anh, HD.; Wataru, K. Cognitive workload detection from raw EEG-signals of vehicle driver using deep learning. IEEE 2019 21st International Conference on Advanced Communication Technology (ICACT), PyeongChang, Korea (South), 17-20 Feb. 2019.
[4] Dehais, F.; Duprès, A., Blum, S.; Drougard, N.; Scannella, S.; Raphaë11e, NR.; Lotte, F. Monitoring pilot's mental workload using ERPs and spectral power with a six-dry-electrode EEG system in real flight conditions. Sensors 2019, 9 (6), 1324.
[5] Anthony, A.; Elizabeth, C.; Prithima, M.; Lukasz, M. Classification of EEG Features for Prediction of Working Memory Load. In Advances in The Human Side of Service Engineering.; Ahram, T., Karwowski, W., Eds.; Springer: Cham, Switzerland, 2017; pp. 32-58.
[6] Zak, Y.; Parmet, Y.; Oron-Gilad, T. Subjective Workload assessment technique (SWAT) in real time: affordable methodology to continuously assess human operators' workload. IEEE 2020 IEEE International Conference on Systems, Man, and Cybernetics (SMC), Toronto, Canada, 17-20 Oct. 2020.
[7] Malagoli, A.; Corradini, M.; Corradini, P.; Shuett, T.; Fonda, S.; Towards a method for the objective assessment of cognitiveworkload: A pilot study in vessel traffic service (VTS) of maritime domain. IEEE 2017 IEEE 3rd International Forum on Research and Technologies for Society and Industry (RTSI), Modena, Italy, 11-13 Sep. 2017.
[8] Vidulich, M. A. (1988). The cognitive psychology of subjective mental workload. In Advances in Psychology.; Peter, A. H., Najmedin, M., Eds.; Elsevier: Amsterdam, The Netherlands, 1988; pp. 219-229.
[9] Antonenko, P.; Paas, F.; Grabner, R.; Van Gog, T. Using electroencephalography to measure cognitive load. Educational psychology review 2010, 22 (4), 425-438.
[10] Mohanavelu, K.; Poonguzhali, S.; Ravi, D.; Singh, PK.; Mahajabin, M.; Ramachandran, K.; Singh, UK.; Jayaraman, S. Cognitive Workload Analysis of Fighter Aircraft Pilots in Flight Simulator Environment. Defence Science Journal 2020, 70 (2), 131-139.
[11] Tao, D.; Tan, H.; Wang, H.; Zhang, X.; Qu, X.; Zhang, T. A systematic review of physiological measures of mental workload. International journal of environmental research and public health 2019, 16 (15), 2716.
[12] Kim, J.; Lee, J.; Han, C.; Park, K. An instant donning multi-channel EEG headset (with comb-shaped dry electrodes) and BCI applications. Sensors 2019, 19 (7), 1537.
[13] Pignoni, G.; Komandur, S. (2019 July). Development of a quantitative evaluation tool of cognitive workload in field studies through eye tracking. In International Conference on Human-Computer Interaction, Orlando, Florida, USA, 26-31 Jul. 2019.
[14] Rozado, D.; Dunser, A. Combining EEG with pupillometry to improve cognitive workload detection. Computer 2015, 48 (10), 18-25.
[15] Aeschbacher, S.; Bossard, M.; Ruperti Repilado, F. J.; Good, N.; Schoen, T.; Zimny, M.; Probst-Hensch, N. M.; Schmidt-Trucksäss, A.; Risch, M.; Risch, L.; Conen, D. Healthy lifestyle and heart rate variability in young adults. European journal of preventive cardiology 2016, 23 (10), 1037-1044.
[16] Shaffer, F.; McCraty, R.; Zerr, C. L. A healthy heart is not a metronome: an integrative review of the heart's anatomy and heart rate variability. Frontiers in psychology 2014, 5, 1040.
[17] Malliani, A.; Pagani, M.; Lombardi, F.; Cerutti, S. Cardiovascular neural regulation explored in the frequency domain. Circulation 1991, 84 (2), 482-492.
[18] Mansikka, H.; Virtanen, K.; Harris, D.; Simola, P. Fighter pilots' heart rate, heart rate variation and performance during an instrument flight rules proficiency test. Applied ergonomics 2016, 56, 213-219.
[19] Fujiwara, K.; Abe, E.; Kamata, K.; Nakayama, C.; Suzuki, Y.; Yamakawa, T.; Hiraoka, T.; Kano, M.; Sumi, Y.; Masuda, F.; Matsuo, M. Heart rate variability-based driver drowsiness detection and its validation with EEG. IEEE Transactions on Biomedical Engineering 2018, 66 (6), 1769-1778.
[20] Tjolleng, A.; Jung, K.; Hong, W.; Lee, W.; Lee, B.; You, H.; Son, J.; Park, S. Classification of a Driver's cognitive workload levels using artificial neural network on ECG signals. Applied ergonomics 2017, 59, 326-332.
[21] Kalevo, L.; Miettinen, T.; Leino, A.; Kainulainen, S.; Korkalainen, H.; Myllymaa, K.; Töyras, J.; Leppänen, T.; Laitinen, T.; Myllymaa, S. (2020). Effect of sweating on electrode-skin contact impedances and artifacts in EEG recordings with various screen-printed Ag/Agcl electrodes. IEEE Access 2020, 8, 50934-50943.
[22] Rodrigues, E.; Lima, D.; Barbosa, P.; Gonzaga, K.; Guerra, R. O.; Pimentel, M.; Barbosa, H.; Maciel, A. HRV Monitoring Using Commercial Wearable Devices as a Health Indicator for Older Persons during the Pandemic. Sensors 2022, 22 (5), 2001.
[23] Zhu, K.; Kiourti, A. A Review of Magnetic Field Emissions from the Human Body: Sources, Sensors, and Uses. IEEE Open Journal of Antennas and Propagation 2022, 3, 732-744.
[24] Zhu, K.; Shah, A. M.; Berkow, J.; Kiourti, A. Miniature Coil Array for Passive Magnetocardiography in Non-Shielded Environments. IEEE Journal of Electromagnetics, RF and Microwaves in Medicine and Biology 2020, 5 (2), 124-131.
[25] Bauemschmitt, R.; Feuerstein, M.; Schirmbeck, E. U.; Traub, J.; Klinker, G.; Wildhirt, S. M.; Lange, R. Improved preoperative planning in robotic heart surgery. In Computers in Cardiology 2004, 773-776.
[26] Turner, J. R.; Hewitt, J. K.; Morgan, R. K.; Sims, J.; Carroll, D.; Kelly, K. A. Graded mental arithmetic as an active psychological challenge. International journal of psychophysiology: official journal of the International Organization of Psychophysiology 1986, 3 (4), 307-309.
[27] Novak, D. Biomechatronic applications of brain-computer interfaces. In Handbook of biomechatronics: 1st ed.; Jacob S. Eds.; Academic Press: Cambridge, United States, 2019; pp. 129-175.
[28] Mohanavelu, K.; Poonguzhali, S.; Ravi, D.; Singh, P. K.; Mahajabin, M.; Ramachandran, K.; Singh, U.K.; Jayaraman, S. Cognitive Workload Analysis of Fighter Aircraft Pilots in Flight Simulator Environment. Defence Science Journal 2020, 70 (2), 131-139.
[29] Kurosaka, C. E.; Kuraoka, H.; Miyake, S. Poincaré Plot Indexes of Heart Rate Variability: Pattern II Responses and Mental Workload. International Conference on Human-Computer Interaction, Springer: Cham, Switzerland; 2019; pp. 238-243.
[30] Rosu, G.; Rau, M. C.; Baltag, O. Comparison of signal processing methods applied on a magnetocardiographic signal. In 2017 10th International Symposium on Advanced Topics in Electrical Engineering (ATEE), Bucharest, Romania, 23-25 Mar. 2017; pp. 248-253.
[31] Friedman, B. H.; Allen, M. T.; Christie, I. C.; Santucci, A. K. Validity concerns of common heart-rate variability indices. IEEE Engineering in Medicine and Biology Magazine/2022, 21 (4), 35-40.
[32] Kim, G. M.; Woo, J. M. Determinants for heart rate variability in a normal Korean population. Journal of Korean medical science 2011, 26 (10), 1293-1298.
[33] Sammito, S.; Böckelmann, I. Reference values for time- and frequency-domain heart rate variability measures. Heart Rhythm 2016, 13 (6), 1309-1316.
[34] Ernst, G. Heart-rate variability—more than heart beats? Frontiers in public health 2017, 5, 240.
[35] Reermann, J.; Elzenheimer, E.; Schmidt, G. Real-time biomagnetic signal processing for uncooled magnetometers in cardiology. IEEE Sensors Journal 2019, 519 (11), 4237-4249.
[36] Lanfranchi, P. A.; Somers, V. K. Cardiovascular physiology: autonomic control in health and in sleep disorders. In Principles and Practice of Sleep Medicine: 5th ed.; Meir H. K., Thomas, R., William C. D. Eds.; Elsevier Inc.: Philadelphia, United States, 2010; Volume 1, pp. 226-236.
[37] Goldberger, J. J.; Challapalli, S.; Tung, R.; Parker, M. A.; Kadish, A. H. Relationship of heart rate variability to parasympathetic effect. Circulation 2001, 103 (15), 1977-1983.
[1A] Fenici, R. R.; Melillo, G. Biomagnetically localizable multipurpose catheter and method for MCG guided intracardiac electrophysiology, biopsy and ablation of cardiac arrhythmias. Int. J. Card. Imaging 1991, 7, 207-215.
[2A] Dutz, S.; Bellemann, M. E.; Leder, U.; Haueisen, J. Passive vortex currents in magneto- and electrocardiogramparison of magnetic and electric signal strengths. Phys. Med. Biol. 2005, 51, 145-151.
[3A] Hu, Z.; Ye, K., Bai, M.; Yang, Z.; Lin, Q. Solving the Magnetocardiography Forward Problem in a Realistic Three-Dimensional Heart-Torso Model. IEEE Access 2021, 9, 107095-107103.
[4A] Zhu, K.; Kiourti, A. A review of magnetic field emissions from the human body: Sources, sensors, and uses. IEEE Open J. Antennas Propag. 2022, 3, 732-744.
[5A] Sriram, B.; Mencer, M. A.; McKelvey, S.; Siegel, E. R.; Vairavan, S.; Wilson, J. D.; Preissl, H.; Eswaran, H.; Govindan, R. B. Differences in the sleep states of IUGR and low-risk fetuses: An MCG study. Early Hum. Dev. 2013, 89, 815-819.
[6A] Collet, J. P.; Thiele, H.; Barbato, E.; Barthélémy, O.; Bauersachs, J.; Bhatt, D. L.; Dendale, P.; Dorobantu, M.; Edvardsen, T.; Folliguet, T.; et al. 2020 ESC guidelines for the management of acute coronary syndromes in patients presenting without persistent ST-segment elevation. Eur. Heart J. 2020, 42, 1289-1367.
[7A] Iwakami, N.; Aiba, T.; Kamakura, S.; Takaki, H.; Furukawa, T. A.; Sato, T.; Sun, W.; Shishido, T.; Nishimura, K.; Yamada-Inoue, Y.; et al. Identification of malignant early repolarization pattern by late QRS activity in high-Resolution Magnetocardiography. Ann. Noninvasive Electrocardiol. 2020, 25, 4.
[8A] Fenici, R.; Brisinda, D.; Meloni, A. M. Clinical application of magnetocardiography. Exp. Rev. Mol. Diagn. 2005, 5, 291-313.
[9A] Nenonen, J.; Nenonen, B. Simulation of extracardiac electromagnetic field due to propagated excitation in the anisotropic ventricular myocardium. In Biomedical and Life Physics; Ghista, D., Ed.; Viewer Verlag: Berlin, Germany, 1996; pp. 191-202.
[10A] Li, H.; Zhang, S.; Zhang, C.; Xie, X. Squid-based MCG measurement using a full-tensor compensation technique in an urban hospital environment. IEEE
Trans. Appl. Supercond. 2016, 26, 1-5.
[11A] Romalis, M. V.; Dang, H. B. Atomic magnetometers for materials characterization. Mater. Today 2011, 14, 258-262.
[12A] Oelsner, G.; Jsselsteijn, R. I.; Scholtes, T.; Krüger, A.; Schultze, V.; Seyffert, G.; Werner, G.; Jäger, M.; Chwala, A.; Stolz, R. Integrated optically pumped magnetometer for measurements within Earth's magnetic field. Phys. Rev. Appl. 2022, 17, 2.
[13A] Sutter, J. U.; Lewis, O.; Robinson, C.; McMahon, A.; Boyce, R.; Bragg, R.; Macrae, A.; Orton, J.; Shah, V.; Ingleby, S. J.; et al. Recording the heart beat of cattle using a gradiometer system of optically pumped magnetometers. Comput. Electron. Agric. 2020, 177, 105651.
[14A] Mooney, J. W.; Ghasemi-Roudsari, S.; Banham, E. R.; Symonds, C.; Pawlowski, N.; Varcoe, B. T. A portable diagnostic device for cardiac magnetic field mapping. Biomed. Phys. Eng. Express 2017, 3, 015008.
[15A] Kurashima, K.; Kataoka, M.; Nakano, T.; Fujiwara, K.; Kato, S.; Nakamura, T.; Yuzawa, M.; Masuda, M.; Ichimura, K.; Okatake, S.; et al. Development of magnetocardiograph without magnetically shielded room using high-detectivity TMR sensors. Sensors 2023, 23, 646.
[16A] Zhu, K.; Shah, A. M.; Berkow, J.; Kiourti, A. Miniature coil array for passive magnetocardiography in non-shielded environments. IEEE J. Electromagn. RF Microw. Med. Biol. 2021, 5, 124-131.
[17A] Zhu, K.; Kiourti, A. Air-core coil gradiometer for biomagnetic sensing in non-shielded environments. In Proceedings of the 2021 United States National Committee of URSI National Radio Science Meeting (USNC-URSI NRSM), Boulder, CO, USA, 4-9 Jan. 2021.
[18A] Zhu, K.; Kiourti, A. Detection of extremely weak and wideband bio-magnetic signals in non-shielded environments using passive coil sensors. IEEE J. Electromagn. RF Microw. Med. Biol. 2022, 6, 501-508.
[19A] Elekta. Elekta Technology Virtually Eliminates MEG Helium Refills. Elekta AB. Available online: http://www.elekta.com/meta/press-intern/?id=c9c8fa2c-349a-4835-bd8b-78231ec2729c (accessed on 29 Jan. 2020).
[20A] Katsigiannis, S.; Ramzan, N. Dreamer: A database for emotion recognition through EEG and ECG signals from wireless low-cost off-the-shelf devices. IEEE J. Biomed. Health Inform. 2018, 22, 98-107.
[21A] Ehmen, H.; Haesner, M.; Steinke, I.; Dorn, M.; Gövercin, M.; Steinhagen-Thiessen, E. Comparison of four different mobile devices for measuring heart rate and ECG with respect to aspects of usability and acceptance by older people. Appl. Ergon. 2012, 43, 582-587.
[22A] Sun, F.-T.; Kuo, C.; Griss, M. Pear: Power efficiency through activity recognition (for ECG-based sensing). In Proceedings of the 5th International ICST Conference on Pervasive Computing Technologies for Healthcare, Dublin, Ireland, 23-26 May 2011.
[23A] Yakut, O.; Solak, S.; Bolat, E. D. Measuring ECG Signal Using e Health Sensor Platform. In Proceedings of the International Conference on Chemistry, Biomedical and Environment Engineering (ICCBEE′14), Antalya, Turkey, 7-8 Oct. 2014.
[24A] Kher, R. Signal processing techniques for removing noise from ECG signals. J. Biomed. Eng. Res. 2019, 3, 1-9.
[25A] Chatterjee, S.; Thakur, R. S.; Yadav, R. N.; Gupta, L.; Raghuvanshi, D. K. Review of noise removal techniques in ECG signals. IET Signal Process. 2020, 14, 569-590.
[26A] Goldberger, A. L.; Goldberger, Z. D.; Shvilkin, A. How to make basic ECG measurements. In Goldberger's Clinical Electrocardiogramplified Approach; Elsevier/Saunders: Philadelphia, PA, USA, 2018; pp. 12-21.
[27A] Clifford, G. D. ECG Statistics, Noise, Artifacts, and Missing Data. In Advanced Methods and Tools for ECG Data Analysis; Artech House: Boston, MA, USA, 2006; pp. 55-99.
[28A] Kandori, A.; Ogata, K.; Watanabe, Y.; Takuma, N.; Tanaka, K.; Murakami, M.; Miyashita, T.; Sasaki, N.; Oka, Y. Space-time database for standardization of adult magnetocardiogram-making standard MCG parameters. Pacing Clin. Electrophysiol. 2008, 31, 422-431.
[29A] Kandori, A.; Ogata, K.; Miyashita, T.; Watanabe, Y.; Tanaka, K.; Murakami, M.; Oka, Y.; Takaki, H.; Hashimoto, S.; Yamada, Y.; et al. Standard template of Adult Magnetocardiogram. Ann. Noninvasive Electrocardiol. 2008, 13, 391-400.
[30A] Faley, M. I.; Poppe, U.; Urban, K.; Slobodchikov, V. Y.; Maslennikov, Y. V.; Gapelyuk, A.; Sawitzki, B.; Schirdewan, A. Operation of high-temperature superconductor magnetometer with submicrometer Bicrystal Junctions. Appl. Phys. Lett. 2002, 81, 2406-2408.
[31A] Zhang, Y.; Wolters, N.; Schubert, J.; Lomparski, D.; Banzet, M.; Panaitov, G.; Krause, H. J.; Muck, M.; Braginski, A. I. HTS SQUID gradiometer using substrate resonators operating in an unshielded environment-A portable MCG system. IEEE Trans. Appl. Supercond. 2003, 13, 389-392.
[32A] Yabukami, S.; Kato, K.; Ohtomo, Y.; Ozawa, T.; Arai, K. I. A thin film magnetic field sensor of sub-pt resolution and magnetocardiogram (MCG) measurement at room temperature. J. Magn. Magn. Mater. 2009, 321, 675-678.
[33A] Uchiyama, T.; Takiya, T. Development of precise off-diagonal magnetoimpedance gradiometer for magnetocardiography. AIP Adv. 2017, 7, 056644.
[34A] Wang, Z.; Xu, M.; Xu, X.; Zhou, Z. Bio-magnetic sensor circuit design based on giant magneto-impedance effect. In Proceedings of the 2016 IEEE International Conference on Mechatronics and Automation, Harbin, China, 7-10 Aug. 2016.
[35A] Nair, V. V.; Youn, J. H.; Choi, J. R. An integrated chip coil sensor and instrumentation amplifier for bio-magnetic signal acquisition In Proceedings of the 2015 Transducers-2015 18th International Conference on Solid-State Sensors, Actuators and Microsystems (TRANSDUCERS), Anchorage, AK, USA, 21-25 Jun. 2015.
[36A] Han, C.; Xu, M.; Tang, J.; Liu, Y.; Zhou, Z. Giant magneto-impedance sensor with working point selfadaptation for unshielded human bio-magnetic detection. Virtual Real. Intell. Hardw. 2022, 4, 38-54.

What is claimed:

1. A method comprising:
receiving a cardiac signal measured by a non-contact sensor;
determining a heart rate variability (HRV) metric based on the cardiac signal; and
determining a cognitive load of a subject based on the HRV metric.

2. The method of claim 1, wherein the non-contact sensor is a magnetocardiography (MCG) sensor.

3. The method of claim 1 or claim 2, wherein the non-contact sensor is a wearable sensor.

4. The method of any one of claims 1-3, wherein determining the HRV metric comprises evaluating the cardiac signal in a time domain.

5. The method of any one of claims 1-4, wherein the HRV metric is a standard deviation of RR intervals (SDRR) in the cardiac signal, a root mean square of successive differences between heartbeats (RMSSD) in the cardiac signal, or a mean value of adjacent R-peaks (MeanRR) in the cardiac signal.

6. The method of any one of claims 1-3, wherein determining the HRV metric comprises evaluating the cardiac signal in a frequency domain.

7. The method of any one of claims 4-6, wherein determining the cognitive load of the subject comprises evaluating the HRV metric using a Poincaré plot.

8. The method of any one of claims 1-7, wherein the non-contact sensor comprises a belt and a plurality of coils, wherein the coils are embedded in the belt.

9. The method of any one of claims 1-8, wherein determining the cognitive load of the subject comprises distinguishing between high and low cognitive load based on the HRV metric.

10. The method of any one of claims 1-8, wherein determining the cognitive load of the subject comprises classifying the subject into one of a plurality of cognitive load categories based on the HRV metric.

11. The method of any one of claims 1-8, wherein determining the cognitive load of the subject comprises quantifying the cognitive load of the subject based on the HRV metric.

12. The method of any one of claims 1-11, wherein determining the HRV metric comprises identifying R-peaks, identifying a plurality of MCG cycles using the R-peaks, and averaging the plurality of MCG cycles to obtain an MCG waveform.

13. A system comprising:
a non-contact sensor; and
a computing device operably coupled to the non-contact sensor, the computing device comprising a processor and a memory, the memory having computer-executable instructions thereon, that, when executed, cause the processor to:
receive a cardiac signal measured by the non-contact sensor;
determine a heart rate variability (HRV) metric based on the cardiac signal; and
determine a cognitive load of a subject based on the HRV metric.

14. The system of claim 13, wherein the non-contact sensor is a magnetocardiography (MCG) sensor.

15. The system of any one of claims 13-14, wherein the non-contact sensor is a wearable sensor.

16. The system of any one of claims 13-15, wherein determining the HRV metric comprises evaluating the cardiac signal in a time domain.

17. The system of any one of claims 13-16, wherein the HRV metric is a standard deviation of RR intervals (SDRR) in the cardiac signal, a root mean square of successive differences between heartbeats (RMSSD) in the cardiac signal, or a mean value of adjacent R-peaks (MeanRR) in the cardiac signal.

18. The system of any one of claims 13-15, wherein determining the HRV metric comprises evaluating the cardiac signal in a frequency domain.

19. The system of any one of claims 16-18, wherein determining the cognitive load of the subject comprises evaluating the HRV metric using a Poincaré plot.

20. The system of any one of claims 13-19, wherein the non-contact sensor comprises a belt and a plurality of coils, wherein the coils are embedded in the belt.

21. The system of any one of claims 13-20, wherein determining the cognitive load of the subject comprises distinguishing between high and low cognitive load based on the HRV metric.

22. The system of any one of claims 13-21, wherein determining the cognitive load of the subject comprises classifying the subject into one of a plurality of cognitive load categories based on the HRV metric.

23. The system of any one of claims 13-22, wherein determining the cognitive load of the subject comprises quantifying the cognitive load of the subject based on the HRV metric.

24. The system of any one of claims 13-23, wherein determining the HRV metric comprises identifying R-peaks, identifying a plurality of MCG cycles using the R-peaks, and averaging the plurality of MCG cycles to obtain an MCG waveform.
